(19) United States
(12) Patent Application Publication
(10) Pub. No.: US 2025/0114337 A1
(43) Pub. Date: Apr. 10, 2025

(54) METHODS FOR VASCULAR REGENERATION AND WOUND TREATMENT

(71) Applicant: THE METHODIST HOSPITAL, Houston, TX (US)

(72) Inventors: John P. Cooke, Houston, TX (US); Li Lai, Houston, TX (US)

(21) Appl. No.: 18/730,105

(22) PCT Filed: Jan. 23, 2023
(86) PCT No.: PCT/US2023/061112

§ 371 Date: Jul. 18, 2024

Related U.S. Application Data
(60) Provisional application No. 63/301,715, filed on Jan. 21, 2022.

(51) Int. Cl. A61K 31/429; A61K 31/711; A61K 31/7115; A61K 45/06; A61P 9/10

(52) U.S. Cl. CPC A61K 31/429; A61K 31/711; A61K 31/7115; A61K 45/06; A61P 9/10

(57) ABSTRACT

Described are methods for vascular regeneration m a subject with peripheral vascular disease. Described are also methods of treating a wound. The methods can include administering an effective amount of an O-glycnacylation modifier agent described here. The method increases O-glycnacylation level in the wound compared to O-glycnacylation level without administration of an effective amount of an O-glycnacylation modifier agent.

### CROSS-REFERENCE TO RELATED APPLICATIONS

This application claims the benefit of U.S. Provisional Application No. 63/301,715, filed Jan. 21, 2022, which is hereby incorporated herein by reference in its entirety.

### STATEMENT REGARDING FEDERALLY SPONSORED RESEARCH OR DEVELOPMENT

This invention was made with government support under Grant Nos. HL133254 and HL148338 awarded by National Institutes of Health. The government has certain rights in the invention.

### INCORPORATION BY REFERENCE

The content of the XML filed named “10063-069WO1_2023_01_23_Sequence_Listing.xml” which was created on Jan. 20, 2023, and is 12,337 bytes in size, is hereby incorporated by reference in their entirety.

### BACKGROUND

Shinya Yamanaka found that retroviral expression of transcriptional factors Oct 4, Sox2, KLF4 and c-Myc (OSKM) in fibroblasts can induce pluripotency. Nuclear reprogramming of fibroblasts to pluripotency was shown to require inflammatory signaling. in addition to OSKM (Lee, J., et al., Cell, 2012. 151 (3): p. 547-58 (“Lee, et al.,”)), the retroviral vector used by Yamanaka played a role in cell fate transition by activating inflammatory signaling (Lee, et al.; Chanda, P. K., et al., Circulation, 2019. 140 (13): p. 1081-1099 (“Chanda, et al.”)). Subsequently, it was shown that other changes in cell fate, such as transdifferentiation of fibroblasts to endothelial cells, required inflammatory signaling (as well as appropriate transcriptional cues) (Sayed, N., et al., Circulation, 2015. 131 (3): p. 300-9). This process of “transflammation” is associated with global changes in the expression and activity of epigenetic modifiers (Chanda, et al.; Meng, S., et al., Circ Res, 2016. 119 (9): p. e129-e138). These epigenetic changes increase DNA accessibility to facilitate cellular plasticity leading to changes in cell fate (Lee, et al.; Chanda, et al.). Recent experiments using fibroblast-specific lineage-tracing mice showed that this process is also activated with tissue ischemia in vivo (Meng, S., et al., Circulation, 2020. 142 (17): p. 1647-1662). Lineage tracing combined with single cell analysis revealed a subset of fibroblasts in the mouse hindlimb that are poised for transdifferentiation into endothelial cells. In vivo, these fibroblasts express low levels of endothelial genes. When isolated from the limb, this fibroblast subset transforms into endothelial cells in Matrigel (which contains endothelial growth factors). In addition to inflammatory signaling, studies revealed that a glycolytic shift was also required for the increase in DNA accessibility (Lai, L., et al., Circulation, 2019. 139 (1): p. 119-133). Most recently, it was determined, that uridine diphosphate N-acetylglucosamine (UDP-GlcNAc) and O-GlycNAcylation are increased during the recovery from in vivo limb ischemia. It was determined that this metabolic process is required for fibroblast to endothelial cell transdifferentiation in vitro.

Critical limb ischemia (CLI), the severe form of peripheral artery disease, accounts for 12% of the U.S. adult population (Roth, G. A., et al., J Am Coll Cardiol, 2020. 76 (25): p. 2982-3021) with a mortality rate of 20% to 26% within 1 year of diagnosis (Conte, M. S., et al., Eur J Vasc Endovasc Surg, 2019. 58 (1S): p. S1-S109 e33). One of the few treatments is amputation suggesting a huge clinical need for efficacious treatments (Cooke, J. P., et al., Circ Res, 2015. 116 (9): p. 1561-78). Ischemia-induced neovascularization is critical for perfusion recovery (Cooke, J. P. et al., Arterioscler Thromb Vasc Biol, 2020. 40 (7): p. 1627-1634); however, no known medication can induce enough functional blood vessel growth and thus treat CLI (Annex, B. H. et al., Circ Res, 2021. 128 (12): p. 1944-1957; Annex, B. H., Nat Rev Cardiol, 2013. 10 (7): p. 387-96).

There is a need for an enhanced transdifferentiation of fibroblasts to endothelial cells for vascular regeneration. The compositions and methods disclosed herein address these and other needs.

### SUMMARY

Provided herein are methods for vascular regeneration in a subject with peripheral vascular disease, the method can include administering an effective amount of an O-glycnacylation modifier agent to an injured peripheral vascular tissue in the subject.

In some embodiments, the methods can further include administering an effective amount of an inflammation agent inducer, an angiogenic factor, or any combination thereof.

In some embodiments, the method can promote vascular regeneration in the injured peripheral vascular tissue by at least 30% compared to the peripheral vascular tissue without administration of an effective amount of an O-glycnacylation modifier agent, as determined by laser doppler perfusion.

In some embodiments, the method can increase O-glycnacylation level in the injured peripheral vascular tissue compared to O-glycnacylation level without administration of an effective amount of an O-glycnacylation modifier agent. In some embodiments, the method can increase the concentration of O-GlycNAc transferase (OGT) in the injured peripheral vascular tissue compared to the concentration O-GlycNAC transferase (OGT) without administration of an effective amount of an O-glycnacylation modifier agent. In some embodiments, the method can inhibit O-GlycNACase (OGA) level in the injured peripheral vascular tissue compared to the O-GlycNACase (OGA) level without administration of an effective amount of an O-glycnacylation modifier agent. In some embodiments, the method can increase cellular levels of UDP-GlcNAc up to 4-fold as determined by metabolomic studies of fibroblasts.

In some embodiments, the peripheral vascular disease can include peripheral arterial disease, limb ischemia, popliteal entrapment syndrome, Raynaud's disease, Buerger's disease, or any combination thereof. In some embodiments, the peripheral vascular disease is peripheral arterial occlusive disease. In some embodiments, the peripheral vascular disease is associated with limb ischemia.

In some embodiments, the peripheral vascular tissue can include cutaneous tissue, epithelial tissue, connective tissue, muscle tissue, bone, nervous tissue, or any combination thereof.

Described herein are also methods of treating a wound in a subject in need thereof, the method can include administering an effective amount of an O-glycnacylation modifier agent to the wound in the subject. In some embodiments, the methods can further include administering an effective amount of an inflammation agent inducer, an angiogenic factor, or any combination thereof.

In some embodiments, the method can promote wound healing by an amount of from 5% to 50% compared to the wound healing without administration of an effective amount of an O-glycnacylation modifier agent.

In some embodiments, the method increases O-glycnacylation level in the wound compared to O-glycnacylation level without administration of an effective amount of an O-glycnacylation modifier agent. In some embodiments, the method can increase the concentration O-GlycNAC transferase (OGT) in the wound compared to the concentration O-GlycNAC transferase (OGT) without administration of an effective amount of an O-glycnacylation modifier agent. In some embodiments, the method inhibits O-GlycNACase (OGA) level in the wound compared to the O-GlycNACase (OGA) level without administration of an effective amount of an O-glycnacylation modifier agent. In some embodiments, the method can increase cellular levels of UDP-GlcNAc up to 4-fold as determined by metabolomic studies of fibroblasts.

In some embodiments, the wound can be a vascular wound. In some embodiments, the wound can be a surgical wound. In some embodiments, the wound can be present on a limb and extremities. In some embodiments, the wound can be a non-healing wound. Non-healing wounds refer to wounds that fail to progress in a timely manner, usually within a timeframe of 4 weeks to 3 months (e.g., from 1 month to 2 months, from 2 months to 3 months, or from 1.5 months to 2.5 months).

In some embodiments, the O-glycnacylation modifier agent can include 2-(ethylamino)-5-(hydroxymethyl)-5,6,7,7a-tetrahydro-3aH-pyrano[3,2-d][1,3]thiazole-6,7-diol (TMG); (3aR,5R,6S,7R,7aR)-3a,6,7,7a-Tetrahydro-5-(hydroxymethyl)-2-propyl-5H-pyrano[3,2-d]thiazole-6,7-diol (NButGT); NAG-thiazoline (i.e. 2′-methyl-a-D-glucopyrano-[2,1-i/]-A2′-thiazoline); O-(2-acetamido-2-deoxy-D-glucopyranosylidene)amino-Z—N-phenylcarbamate) (PUGNAc); a polynucleotide sequence encoding O-GlycNAC transferase (OGT), a fragment, or variant thereof; uridine diphosphate N-acetylglucosamine (UDP-GlcNAc); glucose; glutamine; glucosamine; or any combination thereof. In some embodiments, the O-glycnacylation modifier agent can include 2-(ethylamino)-5-(hydroxymethyl)-5,6,7,7a-tetrahydro-3aH-pyrano[3,2-d][1,3]thiazole-6,7-diol (TMG). In some embodiments, the inflammation agent inducer can include TLR3 agonist polyinosinic; polycytidilic acid (PolyIC). In some embodiments, the angiogenic factor can include VEGF.

In some embodiments, the polynucleotide sequence can encode O-GlycNAC transferase, a fragment, or a variant including an amino acid sequence with at least 80%, at least 85%, at least 90%, at least 95%, at least 96%, at least 97%, at least 98%, at least 99% sequence identity to SEQ ID NO: 2. In some embodiments, the polynucleotide sequence can be an mRNA sequence including a coding region encoding O-GlycNAC transferase a fragment, or a variant. In some embodiments, the polynucleotide sequence can be a DNA sequence including a coding region encoding O-GlycNAC transferase a fragment, or a variant. In one embodiment, the DNA sequence can include a coding region encoding O-GlycNAC transferase, a fragment, or a variant, wherein the DNA sequence includes a sequence with at least 80%, at least 85%, at least 90%, at least 95%, at least 96%, at least 97%, at least 98%, at least 99% sequence identity to SEQ ID NO: 1.

In some embodiments, administration can include topical, intravenous, subcutaneous, transcutaneous, transdermal, intramuscular, intradermal, intra-arteriole, intralesional, or any combination thereof.

The details of one or more embodiments of the disclosure are set forth in the accompanying drawings and the description below. Other features, objects, and advantages of the disclosure will be apparent from the description and drawings, and from the claims.

### DESCRIPTION OF DRAWINGS

FIG. 1 shows a schematic diagram of OGT: O-GlcNAc transferase; OGA: O-GlcNAcase that catalyzes hydrolysis of O-GlcNAc.

FIGS. 2A-2D show iECs generated by in vitro transdifferentiation protocol (2A) and the resulting iECs functionally mimic genuine endothelial cells. In 2B, the iECs are observed to take up acetylated LDL cholesterol, consistent with endothelial function. In 2C, the iECs are seen to form networks in Matrigel, consistent with endothelial function. In 2D, iECs injected into the ischemic hindlimb of the mouse improve limb blood flow to a greater degree than vehicle (Control), and improve blood flow at least as well as genuine human microvascular endothelial cells (HMVECs).

FIG. 3 shows a graph of UDP-GlcNAc level versus time. UDP-GlcNAc is up-regulated after PolyI:C treatment.

FIG. 4 shows PolyI:C induces O-GlcNAcylation augmentation measured by Click-it O-GlcNAc enzymatic labeling and detection assay. N, nucleus; C, cytoplasm; W, whole cell.

FIGS. 5A-5C show the in vitro transdifferentiation is regulated by O-GlcNAcylation manipulation. (5A) shows TMG (OGA inhibitor) increases while DON (GFAT inhibitor) impairs iEC yield; (5B) shows OGA knockdown decreases iEC yield; (5C) shows OGA knockdown efficiency.

FIGS. 6A-6B show ischemia was introduced into 3-month old C57BL/6 mice (n=6). (6A) show doppler flow imaging results showed a very fast blood flow recovery during the first week which almost complete 2-week post-surgery. (6B) western blot using hindlimb tissue protein shows that O-GlcNAcylation and OGT increased in the ischemic side limb (I) compare with the control limb (C) 3 days post-surgery.

FIGS. 7A-7B show graphs of percent recovery versus time for (7A) OSMI and (7B) TMG compared to control. Results show OSMI impairs and TMG enhances revascularization in ischemic hindlimb mice model measured by laser doppler imaging.

FIGS. 8A-8C show innate immune activation enhances HIRA-H3.3 complex integrity by increasing the interaction between OGT and HIRA (8A), HIRA and H3.3 (8B), and H3.3 protein level (8C).

FIGS. 9A-9B show (9A) western blot images showing HIRA knock down impairs decreases H3.3 and (9B) a graph showing HIRA knock down inhibits transdifferentiation in vitro.

FIGS. 10A-10B show ATAC seq and RNA seq data shows that innate immune signaling activation enhances DNA accessibility. Chromatin accessibility mapping is a powerful approach to identify sites of open chromatin (i.e. to infer sites of active transcription). In ATAC-seq, a Tn5 transposase inserts sequencing adapters into accessible DNA (‘tagmentation’). In the left panel, ATAC-seq shows the average chromatin state in fibroblasts (TSS=transcription start site). In the right panel, exposure of the fibroblasts to polyIC increases the average length (KB) of open chromatin. In FIG. 10B, the volcano plot shows those genes that are significantly upregulated (red) and downregulated (blue) in fibroblasts by the method.

FIG. 11 shows lineage tracing strategy using Col1-Cre/ERT: R26R-tdTomato mice.

FIG. 12 shows a graph of percent CD11b−YFP+ cell versus time. iEC population increased rapidly on day 3 and day 7 post-surgery.

FIG. 13 shows western blot image showing H3.3 and OGT level increased in the ischemic limb (I) compared with the control limb (C) on day 3 post-surgery in YFP+ cells.

FIG. 14 shows breeding strategy to knockout OGT or OGA specifically in fibroblast cells in a tamoxifen inducible manner.

FIG. 15 shows a graph of percent recovery per ROI Ischemic/control versus time. OGT knockout in fibroblasts impairs revascularization in ischemic hindlimb mice.

FIG. 16A-16D shows that UDP-GlcNAc level is significantly upregulated during transdifferentiation. FIG. 16A shows a principal component analysis graph showing clear separation in different time point groups and consistency among the triplicates of each time point. FIG. 16B shows a graph of 18 significant up-regulated metabolites (std<0.05) on day 3 in comparison with day 0. FIG. 16C shows a graph of increases on day 1 and day 3. FIG. 16D shows that the HBP pathway metabolites are significantly enriched which confirmed the importance of HBP and O-GlcNAcylation in the transdifferentiation.

FIG. 17A-17D are graphs showing that UDP-GlcNAc is significantly up-regulated during transdifferentiation (N=3) (FIG. 17A); TMG (OGA inhibitor) increases while OSMI (OGT inhibitor) and DON (GFAT inhibitor) impair iEC yield (FIG. 17B); and OGT knockdown in fibroblasts impairs while OGA knockdown enhances iEC yield (N=5) (FIG. 17C and FIG. 17D).

FIG. 18A-18B shows that O-GlcNAcylation is increased during recovery from ischemia. FIG. 18A shows images of Western blotting of hindlimb tissues. FIG. 18B shows images of immunofluorescent staining showing a significant increase in O-GlcNAcylation in the ischemic limb in comparison with the control limb 3 days post-surgery.

FIG. 19A-19D shows O-GlcNAcylation is required for the vascular recovery. FIG. 19A shows a diagram of WT C57BL/6 mice were treated with the O-GlcNAcylation inhibitor, OSMI4 (10 mg/kg), or O-GlcNAcylation enhancer, TMG (10 mg/kg) 0, 1, 2 days post-surgery. Doppler imager was used to monitor the data. FIGS. 19B and 19C show graphs showing that OSMI impaired recovery. FIG. 19D show images of OSMI at day 0 and 21.

FIG. 19E show images of TMG at day 0 and 21, TMG enhanced recovery of blood flow post femoral artery ligation.

FIG. 20A-20D show results that O-GlcNAcylation enhances transdifferentiation in vivo. FIG. 20A shows a diagram of fibroblasts lineage tracing Fsp1-Cre: R26R-EYFP mice strain. FIG. 20B show a graph demonstrating that the YFP+CD31+CD11b-population expanded rapidly on day 3 and day 7 post-surgery. FIG. 20C show immunofluorescent staining images. FIG. 20D shows a graph of western results showing a dramatic accumulation of O-GlcNAcylation in the YFP+ cells, especially in the ischemic limb.

FIG. 21A-21F shows that fibroblast-specific O-GlcNAcylation manipulation regulates vascular recovery. FIG. 21A shows a graph showing that the fibroblast-specific OGT knockout impairs while OGA knockout enhances vascular recovery. FIG. 21B shows a graph showing that the fibroblast-specific OGT knockout impairs while OGA knockout enhances vascular recovery. for male subjects. FIG. 21C shows a graph showing that the fibroblast-specific OGT knockout impairs while OGA knockout enhances vascular recovery. female subjects. FIG. 21D shows a graph of results of CD31 immunofluorescent staining on the limb tissue sections. FIG. 21E shows a graph of results of CD31 immunofluorescent staining on the limb tissue sections for male subjects. FIG. 21F shows a graph of results of CD31 immunofluorescent staining on the limb tissue sections for female subjects.

FIG. 22A-22C shows HIRA-H3.3 interaction is activated during ischemia and vascular recovery. FIG. 22A show western blot images showing the level of HIRA complex subunits including ASFIA, HIRA, UBN, as well as the H3.3 and GAPDH. FIG. 22B shows western blot images of OGlcNAc, OGT, ASFIA, HIRA, UBN, H3.3, and Tubulin in the limbs recovering from ischemia 3- and 7-days post-surgery compared with the non-YFP+ cells. FIG. 22C shows western blot images of UBN, H3.3, HIRA, and GAPDH.

FIG. 23A-23C shows H3.3 deposition is enhanced during transflammation. FIG. 23A shows a diagram of the cell culture and imaging procedure. FIG. 23B shows images of TMR, DAPI, and merge for control, POLY I:C, OSMI, and POLY I:C+OSMI. FIG. 23C shows a graph of change in density for control, poly H3.3b, OSMI H3.3b, and Poly OSMI H3.3b. The results showed that the Poly I:C enhances the H3.3 deposition which is impaired by OSMI (FIG. 23A-23C).

FIG. 24 shows a graph showing that the S231A HIRA mutation overexpressed fibroblasts have impaired transdifferentiation compared with WT HIRA control suggested the O-GlcNAcylation at S231 HIRA is critical for transdifferentiation.

FIG. 25A-25B shows graphs of transcriptional and chromatin accessibility profiling data RNA seq (25A) and ATAC seq (25B).

FIG. 26 shows western blot images of O-GlcNAcylation level, HIRA complex proteins and h3.3 increased in the YFP+ cell 3 days post-surgery in the ischemic limb compare with the control limb.

FIG. 27 shows images of O-GlcNAc, GAPDH, and OGT. O-GlcNAcylation and OGT are upregulated in ischemic tissue from non-PAD patient but not in critical limb ischemia (CLI) patient. (C, control, non-ischemic tissue; I, ischemic tissue)

FIG. 28 shows a diagram of Visualization of SNAP-tagged h3.3 cycling with TMR-Star in Quench-Chase-Pulse experiments.

FIG. 29 shows images of SNAP-h3.3 labeling experiment. P, pulse; Q-P, quench-pulse; Q-C-P, quench-chase-pulse.

Like reference symbols in the various drawings indicate like elements.

### DETAILED DESCRIPTION

A number of embodiments of the disclosure have been described. Nevertheless, it will be understood that various modifications may be made without departing from the spirit and scope of the invention. Accordingly, other embodiments are within the scope of the following claims.

### Definitions

To facilitate understanding of the disclosure set forth herein, a number of terms are defined below. Unless defined otherwise, all technical and scientific terms used herein generally have the same meaning as commonly understood by one of ordinary skill in the art to which this disclosure belongs. Publications cited herein and the materials for which they are cited are specifically incorporated by reference.

### General Definitions

As used in this specification and the following claims, the terms “comprise” (as well as forms, derivatives, or variations thereof, such as “comprising” and “comprises”) and “include” (as well as forms, derivatives, or variations thereof, such as “including” and “includes”) are inclusive (i.e., open-ended) and do not exclude additional elements or steps. For example, the terms “comprise” and/or “comprising.” when used in this specification, specify the presence of stated features, integers, steps, operations, elements, and/or components, but do not preclude the presence or addition of one or more other features, integers, steps, operations, elements, components, and/or groups thereof. Other than where noted, all numbers expressing quantities of ingredients, reaction conditions, geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Accordingly, these terms are intended to not only cover the recited element(s) or step(s), but may also include other elements or steps not expressly recited. Furthermore, as used herein, the use of the terms “a”. “an”, and “the” when used in conjunction with an element may mean “one.” but it is also consistent with the meaning of “one or more.” “at least one.” and “one or more than one.” Therefore, an element preceded by “a” or “an” does not, without more constraints, preclude the existence of additional identical elements.

It is understood that when combinations, subsets, groups, etc. of elements are disclosed (e.g., combinations of components in a composition, or combinations of steps in a method), that while specific reference of each of the various individual and collective combinations and permutations of these elements may not be explicitly disclosed, each is specifically contemplated and described herein.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. A range may be construed to include the start and the end of the range. For example, a range of 10% to 20% (i.e., range of 10%-20%) can includes 10% and also includes 20%, and includes percentages in between 10% and 20%, unless explicitly stated otherwise herein.

As used herein, the terms “may,” “optionally.” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Administration” to a subject includes any route of introducing or delivering to a subject an agent. Administration can be carried out by any suitable route, including topical, intravenous, subcutaneous, transcutaneous, transdermal, intramuscular, intradermal, intra-arteriole, intralesional, or any combination thereof. “Concurrent administration”, “administration in combination”, “simultaneous administration” or “administered simultaneously” as used herein, means that the compounds are administered at the same point in time or essentially immediately following one another. In the latter case, the two compounds are administered at times sufficiently close that the results observed are indistinguishable from those achieved when the compounds are administered at the same point in time. “Local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting.” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

As used herein, the terms “treating” or “treatment” of a subject includes the administration of a drug to a subject with the purpose of preventing, curing, healing, alleviating, relieving, altering, remedying, ameliorating, improving, stabilizing or affecting a disease or disorder, or a symptom of a disease or disorder. The terms “treating” and “treatment” can also refer to reduction in severity and/or frequency of symptoms, elimination of symptoms and/or underlying cause, prevention of the occurrence of symptoms and/or their underlying cause, and improvement or remediation of damage.

By the term “effective amount” of a O-glycnacylation modifier agent, an inflammation agent inducer, and/or an angiogenic factor is meant a nontoxic but sufficient amount of an O-glycnacylation modifier agent, an inflammation agent inducer, and/or an angiogenic factor to provide the desired effect. The amount of O-glycnacylation modifier agent, an inflammation agent inducer, and/or an angiogenic factor that is “effective” will vary from subject to subject, depending on the age and general condition of the subject, the particular agent, and the like. Thus, it is not always possible to specify an exact “effective amount”. However, an appropriate “effective’ amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts. An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

As used herein, the term “pharmaceutically acceptable” component can refer to a component that is not biologically or otherwise undesirable, i.e., the component may be incorporated into a pharmaceutical formulation of the invention and administered to a subject as described herein without causing any significant undesirable biological effects or interacting in a deleterious manner with any of the other components of the formulation in which it is contained. When the term “pharmaceutically acceptable” is used to refer to an excipient, it is generally implied that the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

As used herein, “pharmaceutically acceptable salt” is a derivative of the disclosed compound in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are typical, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxy benzoic, fumaric, toluenesulfonic, methanesulfonic, ethane disulfonic, oxalic, isethionic, HOOC—(CH2)n-COOH where n is 0-4, and the like, or using a different acid that produces the same counterion. Lists of additional suitable salts may be found, e.g., in Remington's Pharmaceutical Sciences, 17th ed., Mack Publishing Company, Easton, Pa., p. 1418 (1985).

A “control” is an alternative subject or sample used in an experiment for comparison purposes. A control can be “positive” or “negative.”

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician. Administration of the therapeutic agents can be carried out at dosages and for periods of time effective for treatment of a subject. In some embodiments, the subject is a human.

The term “nucleic acid” as used herein means a polymer composed of nucleotides, e.g. deoxyribonucleotides or ribonucleotides.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

The term “oligonucleotide” denotes single- or double-stranded nucleotide multimers of from about 2 to up to about 100 nucleotides in length. Suitable oligonucleotides may be prepared by the phosphoramidite method described by Beaucage and Carruthers, Tetrahedron Lett. 22:1859-1862 (1981), or by the triester method according to Matteucci, et al., J. Am. Chem. Soc. 103:3185 (1981), both incorporated herein by reference, or by other chemical methods using either a commercial automated oligonucleotide synthesizer or VLSIPS™ technology. When oligonucleotides are referred to as “double-stranded,” it is understood by those of skill in the art that a pair of oligonucleotides exist in a hydrogen-bonded, helical array typically associated with, for example, DNA. In addition to the 100% complementary form of double-stranded oligonucleotides, the term “double-stranded,” as used herein is also meant to refer to those forms which include such structural features as bulges and loops, described more fully in such biochemistry texts as Stryer, Biochemistry, Third Ed., (1988), incorporated herein by reference for all purposes.

The term “polynucleotide” refers to a single or double stranded polymer composed of nucleotide monomers. The polynucleotide sequence may be modified, for example, to enhance efficacy and/or to reduce immune responsivity, by using, for example, base modifications or end-capping. In other embodiments, an unmodified polynucleotide sequence is used. For example, the polynucleotide can be an RNA sequence or a DNA sequence. In some embodiments, the mRNA can include an optimized codon. By codon optimizing, the formation of secondary structures can be reduced and translational efficiency improved. In certain embodiments, the codon optimization includes GC enrichment of the coding region. In certain embodiments, the codon optimization includes codon quality enrichment of the coding region. In certain aspects, the mRNA can include one or more regions or parts, which act or function as an untranslated region (UTRs) of a gene. UTRs are transcribed but not translated. In mRNA, the 5′ UTR starts at the transcription start site and continues to the start codon but does not include the start codon. The 3′ UTR starts immediately following the stop codon and continues until the transcriptional termination signal. The use of human-derived UTRs may facilitate the expression of the polypeptide in cells. In some embodiments, the polynucleotide comprises at least one chemically modified nucleotide. In some embodiments, the at least one chemically modified nucleotide comprises a chemically modified nucleobase, a chemically modified ribose, a chemically modified phosphodiester linkage, or a combination thereof. In some embodiments, the polynucleotide sequence as used comprise modified nucleosides such as 5-methylcystonsine or psudouridine.

As used herein “modified” refers to a changed state or structure of a molecule of the invention. Molecules may be modified in many ways including chemically, structurally, and functionally. In one embodiment, the polynucleotides of the present invention are “chemically modified” by the introduction of non-natural nucleosides and/or nucleotides, e.g., as it relates to the natural ribonucleotides A, U, G, and C. Modifications of the nucleosides and/or nucleotides as used in the present invention may be naturally occurring (i.e. comprise a nucleotide and/or nucleoside other than the natural ribonucleotides A, U, G, and C) or may be artificial. Non-canonical nucleotides such as the cap structures are not considered “modified” although they differ from the chemical structure of A, G, C, and U ribonucleotides. As used herein, a “structural” modification is one in which two or more linked nucleosides are inserted, deleted, duplicated, inverted or randomized in a polynucleotide without significant chemical modification to the nucleotides themselves. Because chemical bonds will necessarily be broken and reformed to effect a structural modification, structural modifications are of a chemical nature and hence are chemical modifications. However, structural modifications will result in a different sequence of nucleotides. When the polynucleotides of the present invention are chemically and/or structurally modified, the polynucleotides may be referred to as “modified nucleotides”.

The term “polypeptide” refers to a compound made up of a single chain of D- or L-amino acids or a mixture of D- and L-amino acids joined by peptide bonds. A polypeptide is comprised of approximately twenty, standard naturally occurring amino acids, although natural and synthetic amino acids which are not members of the standard twenty amino acids may also be used. The standard twenty amino acids include alanine (Ala, A), arginine (Arg, R), asparagine (Asn, N), aspartic acid (Asp, D), cysteine (Cys, C), glutamine (Gln, Q), glutamic acid (Glu, E), glycine (Gly, G), histidine, (His, H), isoleucine (Ile, I), leucine (Leu, L), lysine (Lys, K), methionine (Met, M), phenylalanine (Phe, F), proline (Pro, P), serine (Ser, S), threonine (Thr, T), tryptophan (Trp, W), tyrosine (Tyr, Y), and valine (Val, V). The terms “polypeptide sequence” or “amino acid sequence” are an alphabetical representation of a polypeptide molecule.

Conservative substitutions of amino acids in proteins and polypeptides are known in the art. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the polypeptides provided herein.

Substantial changes in protein function or immunological identity are made by selecting substitutions that are less conservative, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

A “variant” refers to a molecule substantially similar in structure. Thus, in one embodiment, a variant refers to a protein whose amino acid sequence is similar to a reference amino acid sequence, but does not have 100% identity with the respective reference sequence. The variant protein has an altered sequence in which one or more of the amino acids in the reference sequence is deleted or substituted, or one or more amino acids are inserted into the sequence of the reference amino acid sequence. As a result of the alterations, the variant protein has an amino acid sequence which is at least 60%, 70%, 75%, 80%, 85%, 90%, or 95% identical to the reference sequence. For example, variant sequences which are at least 95% identical have no more than 5 alterations, i.e. any combination of deletions, insertions or substitutions, per 100 amino acids of the reference sequence.

The term “complementary.” refers to the topological compatibility or matching together of interacting surfaces of a probe molecule and its target. Thus, the target and its probe can be described as complementary, and furthermore, the contact surface characteristics are complementary to each other.

The term “hybridization” refers to a process of establishing a non-covalent, sequence-specific interaction between two or more complementary strands of nucleic acids into a single hybrid, which in the case of two strands is referred to as a duplex.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below; or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%)amino acid sequence identity is defined as the percentage of amino acids in a candidate sequence that are identical to the amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) or 10, M=5, N=−4 and a comparison of both strands. For amino acid sequences, the BLASTP program uses as defaults a word length of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

The term “nucleobase” refers to the part of a nucleotide that bears the Watson/Crick base-pairing functionality. The most common naturally-occurring nucleobases, adenine (A), guanine (G), uracil (U), cytosine (C), and thymine (T) bear the hydrogen-bonding functionality that binds one nucleic acid strand to another in a sequence specific manner.

### Method of Use

Described herein are methods for vascular regeneration in a subject with peripheral vascular disease the method can include administering an effective amount of an O-glycnacylation modifier agent to an injured peripheral vascular tissue in the subject.

In some embodiments, the methods can further include administering an effective amount of an inflammation agent inducer, an angiogenic factor, or any combination thereof. In some embodiments, the methods can include administering an effective amount of an O-glycnacylation modifier agent and an inflammation agent inducer to an injured peripheral vascular tissue in the subject. In some embodiments, the methods can include administering an effective amount of an O-glycnacylation modifier agent and an angiogenic factor to an injured peripheral vascular tissue in the subject. In some embodiments, the methods can include administering an effective amount of an O-glycnacylation modifier agent, an inflammation agent inducer and an angiogenic factor to an injured peripheral vascular tissue in the subject.

In some embodiments, the method can promote vascular regeneration in the injured peripheral vascular tissue by at least 30% (e.g., at least 35%, at least 40%, at least 50%, at least 60%, at least 70%, at least 80%, or at least 90%) compared to the peripheral vascular tissue without administration of an effective amount of an O-glycnacylation modifier agent, as determined by laser doppler perfusion. In some embodiments, the method can promote vascular regeneration in the injured peripheral vascular tissue by 95% or less (e.g., 80% or less, 70% or less, 60% or less, 50% or less, 40% or less, or 35% or less). the method can promote vascular regeneration in the injured peripheral vascular tissue by an amount ranging from any of the minimum values described above to any of the maximum values described above. For example, in some embodiments, the method can promote vascular regeneration in the injured peripheral vascular tissue by from 30% to 95%, (e.g., from 30% to 90%, from 30% to 80%, from 30% to 70%, from 30% to 60%, from 30% to 50%, from 30% to 40%, from 40% to 90%, from 40% to 80%, from 40% to 70%, from 40% to 60%, from 40% to 50%, from 50% to 90%, from 50% to 80%, from 50% to 70%, from 50% to 60%, from 60% to 90%, from 60% to 80%, from 60% to 70%, from 70% to 90%, from 70% to 80%, or from 80% to 90%).

In some embodiments, the method can increase O-glycnacylation level in the injured peripheral vascular tissue compared to O-glycnacylation level without administration of an effective amount of an O-glycnacylation modifier agent. In some embodiments, the method can increase the concentration of O-GlycNAc transferase (OGT) in the injured peripheral vascular tissue compared to the concentration O-GlycNAC transferase (OGT) without administration of an effective amount of an O-glycnacylation modifier agent. In some embodiments, the method can inhibit O-GlycNACase (OGA) level in the injured peripheral vascular tissue compared to the O-GlycNACase (OGA) level without administration of an effective amount of an O-glycnacylation modifier agent.

In some embodiments, the method can increase cellular levels of UDP-GlcNAc by 4-fold or less, (e.g., 3-fold or less, 2-fold or less, 1-fold or less) as determined by metabolomic studies of fibroblasts. In some embodiments, the method can increase cellular levels of UDP-GlcNAc by at least 0.5-fold, (e.g., at least 1-fold, at least 2-fold, at least 3-fold) as determined by metabolomic studies of fibroblasts.

The method can increase cellular levels of UDP-GlcNAc ranging from any of the minimum values described above to any of the maximum values described above. For example, in some embodiments, the method can increase cellular levels of UDP-GlcNAc by from 0.5-fold to 4-fold, (e.g., from 0.5-fold to 1-fold, from 0.5-fold to 2-fold, from 0.5-fold to 3-fold, from 1-fold to 2-fold, from 1-fold to 3-fold, from 1-fold to 4-fold, from 2-fold to 3-fold, from 2-fold to 4-fold, or from 3-fold to 4-fold) as determined by metabolomic studies of fibroblasts.

In some embodiments, the peripheral vascular disease comprises peripheral arterial disease, limb ischemia, popliteal entrapment syndrome, Raynaud's disease, Buerger's disease, or any combination thereof.

In some embodiments, the peripheral vascular disease is peripheral arterial disease. In some embodiments, the peripheral vascular disease is peripheral arterial occlusive disease. In some embodiments, the peripheral vascular disease is associated with limb ischemia such as critical limb ischemia or acute limb ischemia. In some embodiments, the peripheral vascular disease is critical limb ischemia. In some embodiments, the peripheral vascular disease is popliteal entrapment syndrome. In some embodiments, the peripheral vascular disease is Raynaud's disease. In some embodiments, the peripheral vascular disease is Buerger's disease.

In some embodiments, the peripheral vascular tissue can include but is not limited to cutaneous tissue, epithelial tissue, connective tissue, muscle tissue, bone, nervous tissue, or any combination thereof. In some embodiments, the peripheral vascular tissue comprises epithelial tissue. In some embodiments, the peripheral vascular tissue comprises connective tissue. In some embodiments, the peripheral vascular tissue comprises muscle tissue. In some embodiments, the peripheral vascular tissue comprises nervous tissue. In some embodiments, the peripheral vascular tissue comprises cutaneous tissue. In some embodiments, the peripheral vascular tissue comprises bone. In some embodiments, the peripheral vascular tissue can include cutaneous tissue, epithelial tissue, connective tissue, muscle tissue, bone, and nervous tissue.

In some embodiments, the method can improve the function of patients with peripheral arterial disease as assessed by treadmill exercise testing, typically using the Skinner Gardner protocol, although other protocols such as the modified Bruce protocol may be used. An improvement (e.g., by at least 10%, at least 20%, at least 30%, or at least 40%) in the patient's absolute claudication time (ACT; the maximal walking distance before the patient stops due to leg pain) or initial claudication time (ICT; the time point at which the person first develops claudication). In some cases, a 6-minute walking test may be substituted for the treadmill test, in which case the maximum distance walked in 6 minutes is improved (e.g., by at least 10%, at least 20%, at least 30%, or at least 40%).

In some embodiments, the method can enhance blood flow in the affected tissue of patients with peripheral arterial disease as determined by perfusion measurement methods. Suitable perfusion measurement methods can include but are not limited to magnetic image resonance, single photon emission computed tomography, venous occlusion plethysmography, duplex ultrasonography, near infrared spectroscopy, doppler flowmetry or tissue clearance of injected radionuclides, or any combination thereof. An enhancement of blood flow may be shown as an increase (e.g., by at least 10%, at least 20%, at least 30%, or at least 40%) over a specified period of time using the perfusion units specific to these tests and adjudicated by independent observers.

In some embodiments, the method can enhance perfusion in patients with peripheral arterial disease as determined by a reduction in morbidities. An enhancement of perfusion may be shown as a reduction in morbidities (e.g., by at least 10%, at least 20%, at least 30%, or at least 40%) such as clinic visits, surgical procedures, infections, and hospitalizations over a specified period of time using endpoints adjudicated by independent observers.

In some embodiments, the method can increase perfusion by enhancing angiogenesis. The enhancement of angiogenesis can be due to an increase in cellular O-glycnacylation facilitating transdifferentiation of fibroblasts to induced endothelial cells.

Described herein are also methods of treating a wound in a subject in need thereof, the method can include administering an effective amount of an O-glycnacylation modifier agent to the wound in the subject. In some embodiments, the methods can further include administering an effective amount of an inflammation agent inducer, an angiogenic factor, or any combination thereof.

In some embodiments, the methods can include administering an effective amount of an O-glycnacylation modifier agent and an inflammation agent inducer to the wound in the subject. In some embodiments, the methods can include administering an effective amount of an O-glycnacylation modifier agent and an angiogenic factor to the wound in the subject. In some embodiments, the methods can include administering an effective amount of an O-glycnacylation modifier agent, an inflammation agent inducer and an angiogenic factor to the wound in the subject.

In some embodiments, the method can promote wound healing by an amount of at least 5% (e.g., at least 10%, at least 20%, at least 30%, or at least 40%), compared to the wound healing without administration of an effective amount of an O-glycnacylation modifier agent. In some embodiments, the method can promote wound healing by an amount of 50% or less (e.g., 40% or less, 30% or less, 20% or less, 10% or less), compared to the wound healing without administration of an effective amount of an O-glycnacylation modifier agent.

The method can promote wound healing by an amount ranging from any of the minimum values described above to any of the maximum values described above. For example, in some embodiments, the method can promote wound healing by an amount of from 5% to 50% (e.g., from 5% to 40%, from 5% to 30%, from 5% to 20%, from 5% to 15%, from 5% to 10%, from 10% to 50%, from 10% to 40%, from 10% to 30%, from 10% to 20%, from 20% to 50%, from 20% to 40%, from 20% to 30%, from 30% to 50%, from 30% to 40%, or from 40% to 50%), compared to the wound healing without administration of an effective amount of an O-glycnacylation modifier agent.

In some embodiments, the method increases O-glycnacylation level in the wound compared to O-glycnacylation level without administration of an effective amount of an O-glycnacylation modifier agent. In some embodiments, the method increases the concentration O-GlycNAC transferase (OGT) in the wound compared to the concentration O-GlycNAC transferase (OGT) without administration of an effective amount of an O-glycnacylation modifier agent. In some embodiments, the method inhibits O-GlycNACase (OGA) level in the wound compared to the O-GlycNACase (OGA) level without administration of an effective amount of an O-glycnacylation modifier agent. In some embodiments, the method can increase cellular levels of UDP-GlcNAc up to 4-fold as determined by metabolomic studies of fibroblasts.

It is to be understood that the term “wound” refers to open and closed wounds in which skin is torn, cut or punctured or where trauma causes a contusion, or any other superficial or other conditions or imperfections on the skin of a patient. A wound can be defined as any damaged region of tissue where fluid may or may not be produced. In addition, a wound or ulceration can be produced by traumatic or pathogenic disruption of an epithelial layer, such as the gastrointestinal, renal, urethral, ureteral epithelium; or by disruption of an endothelial layer, such as the vascular or cardiac endothelium. Examples of such wounds include, but are not limited to, abdominal wounds or other large or incisional wounds, either as a result of surgery, trauma, sternotomies, fasciotomies, or other conditions, dehisced wounds, acute wounds, chronic wounds, subacute and dehisced wounds, traumatic wounds, vascular wounds (e.g., venous ulcer, arterial ulcer), flaps and skin grafts, surgical wounds, lacerations, abrasions, contusions, hematomas, burns, diabetic ulcers, pressure ulcers, stoma, cosmetic wounds, trauma ulcers, neuropathic ulcers, venous ulcer, arterial ulcers, chronic wound, non-healing wounds, or any combination thereof. Wounds may include readily accessible and difficult to access wounds, exposed and concealed wounds, large and small wounds, regular and irregular shaped wounds, planar and topographically irregular, uneven or complex wounds. The wound can be present on a site selected from the torso, limb and extremities such as heel, sacrum, axial, inguinal, shoulder, neck, leg, foot, digit, knee, avilla, arm and forearm, elbow, hand or any combination thereof. In some embodiments, the wound can be a vascular wound. In some embodiments, the wound can be a surgical wound. In some embodiments, the wound can be a venous ulcer. In some embodiments, the wound can be an arterial ulcer. In some embodiments, the wound can be present on a limb and extremities. In some embodiments, the wound can be a non-healing wound. Non-healing wounds refer to wounds that fail to progress in a timely manner, usually within a timeframe of 4 weeks to 3 months (e.g., from 1 month to 2 months, from 2 months to 3 months, or from 1.5 months to 2.5 months). In some embodiments, the wound can exhibit delayed healing. For example, the wound fails to progress in a timely manner, usually within a timeframe of 4 weeks to 3 months (e.g., from 1 month to 2 months, from 2 months to 3 months, or from 1.5 months to 2.5 months).

In some embodiments, the method can enhance wound healing as determined by digital photography and planimetry. An enhancement of wound healing can be shown as a reduction in the surface area of the wound (e.g., by at least 5%, at least 10%, at least 20%, at least 30%, or at least 40%, at least 50%) over a specified period of time compared to the surface area of the wound without administration of an effective amount of an O-glycnacylation modifier agent. An enhancement of wound healing can be shown as a greater percentage of complete wound healing over a specified period of time (e.g., by at least 5%, at least 10%, at least 20%, at least 30%, or at least 40%, at least 50) compared to the wound healing without administration of an effective amount of an O-glycnacylation modifier agent. In some embodiments, the method can enhance wound healing as determined by reduction in pain. An enhancement of wound healing can be shown as a reduction of pain (e.g., by at least 5%, at least 10%, at least 20%, at least 30%, or at least 40%, at least 50) on a validated instrument (e.g. the Likert scale) over a specified period of time compared to the reduction of pain without administration of an effective amount of an O-glycnacylation modifier agent.

In some embodiments, the method can enhance wound healing as determined by a greater reduction in morbidities (e.g., clinic visits, surgical procedures, infections, and hospitalizations). An enhancement of wound healing may be shown as a greater reduction of such morbidities (e.g., by at least 5%, at least 10%, at least 20%, at least 30%, or at least 40%, at least 50) over a specified period of time using endpoints adjudicated by independent observers.

In some embodiments, the method can enhance wound healing as determined by a reduction in the need for skin grafting, or in the amount of donor skin that is required for skin grafting. An enhancement of wound healing may be shown as a reduction of skin (e.g., by at least 5%, at least 10%, at least 20%, at least 30%, or at least 40%, at least 50) as determined by the size of the donor site. In the case of donor skin that is disaggregated prior to application, the enhancement of wound healing may be shown as a reduction in the absolute number of cells (e.g., by at least 5%, at least 10%, at least 20%, at least 30%, or at least 40%, at least 50) that are required for application to fully heal the wound, or to induce a pre-specified amount of healing.

In some embodiments, the method can promote the generation of induced endothelial cells (iECs) from fibroblasts by at least 40% (e.g., at least 50%, at least 60%, at least 70%, at least 80%, or at least 90%) compared to the iECs generated without administration of an effective amount of an O-glycnacylation modifier agent, as determined by fluorescence activated cell sorting analysis of CD31+ cells.

Suitable angiogenic factors can include but are not limited to VEGF, fibroblast growth factor, hypoxia-inducible growth factor, platelet-derived growth factor, bone matrix protein 4, angiopoeitins, nitric oxide or other agents that increase intracellular cGMP, prostacyclin or other agents that increase intracellular cAMP, or any combination thereof.

Suitable inflammation agent inducers can include but are not limited to TLR3 agonist polyinosinic:polycytidilic acid (PolyIC), inflammatory cytokines such as interleukins IL-1a, IL-6 or IL-8, lipopolysaccharide (LPS) or lipoteichoic acid (LTA), tumor necrosis factor alpha, or any combination thereof.

Suitable O-glycnacylation modifier agents can include agents that increase O-glycnacylation levels by increasing the concentration of O-GlycNAC transferase, or inhibiting O-GlycNACase. For example, suitable O-glycnacylation modifier agents can include but are not limited to 2-(ethylamino)-5-(hydroxymethyl)-5,6,7,7a-tetrahydro-3aH-pyrano[3,2-d][1,3]thiazole-6,7-diol (TMG); (3aR,5R,6S,7R,7aR)-3a,6,7,7a-Tetrahydro-5-(hydroxymethyl)-2-propyl-5H-pyrano[3,2-d]thiazole-6,7-diol (NButGT); NAG-thiazoline (i.e. 2 ‘-methyl-a-D-glucopyrano-[2,1-i/]-A2’-thiazoline); O-(2-acetamido-2-deoxy-D-glucopyranosylidene)amino-Z—N-phenylcarbamate) (PUGNAc); a polynucleotide sequence encoding O-GlycNAC transferase (OGT), a fragment, or variant thereof; uridine diphosphate N-acetylglucosamine (UDP-GlcNAc); glucose; glutamine; glucosamine; or any combination thereof. In some embodiments, the O-glycnacylation modifier agent can include 2-(ethylamino)-5-(hydroxymethyl)-5,6,7,7a-tetrahydro-3aH-pyrano[3,2-d][1,3]thiazole-6,7-diol (TMG). In some embodiments, the O-glycnacylation modifier agent can include (3aR,5R,6S,7R,7aR)-3a,6,7,7a-Tetrahydro-5-(hydroxymethyl)-2-propyl-5H-pyrano[3,2-d]thiazole-6,7-diol (NButGT). In some embodiments, the O-glycnacylation modifier agent can include NAG-thiazoline (i.e. 2 ‘-methyl-a-D-glucopyrano-[2,1-i/]-A2’-thiazoline). In some embodiments, the O-glycnacylation modifier agent can include O-(2-acetamido-2-deoxy-D-glucopyranosylidene)amino-Z—N-phenylcarbamate) (PUGNAc). In some embodiments, the O-glycnacylation modifier agents can include a polynucleotide sequence encoding O-GlycNAC transferase (OGT), a fragment, or variant thereof. In some embodiments, the O-glycnacylation modifier agents can include uridine diphosphate N-acetylglucosamine (UDP-GlcNAc), glucose; glutamine; glucosamine; or any combination thereof.

In some embodiments, the polynucleotide sequence encodes O-GlycNAC transferase, a fragment, or a variant comprising an amino acid sequence with at least 80%, at least 85%, at least 90%, at least 95%, at least 96%, at least 97%, at least 98%, at least 99% sequence identity to SEQ ID NO: 2. In some embodiments, the O-GlycNAC transferase, a fragment, or a variant thereof comprises an amino acid sequence with at least 80%. In some embodiments, the O-GlycNAC transferase, a fragment, or a variant thereof comprises an amino acid sequence with at least 90%. In some embodiments, the O-GlycNAC transferase, a fragment, or a variant thereof comprises an amino acid sequence with at least 95%.

In some embodiments, the O-GlycNAC transferase can be SEQ ID NO: 2 (GenBank: U77413.1).

In some embodiments, the polynucleotide sequence encoding O-GlycNAC transferase (OGT) comprises modified nucleosides that increase translational efficiency and/or reduce immunogenicity. In some embodiments, the polynucleotide sequence can be an mRNA sequence comprising a coding region encoding O-GlycNAC transferase a fragment, or a variant. In some embodiments, the polynucleotide sequence can be a DNA sequence comprising a coding region encoding O-GlycNAC transferase a fragment, or a variant. In one embodiment, the DNA sequence comprises a coding region encoding O-GlycNAC transferase, a fragment, or a variant, wherein the DNA sequence comprises a sequence with at least 80%, at least 85%, at least 90%, at least 95%, at least 96%, at least 97%, at least 98%, at least 99% sequence identity to SEQ ID NO: 1.

In some embodiments, the DNA sequence comprises a sequence with at least 80% sequence identity to SEQ ID NO: 1. In some embodiments, the DNA sequence comprises a sequence with at least 90% sequence identity to SEQ ID NO: 1. In some embodiments, the DNA sequence comprises a sequence with at least 95% sequence identity to SEQ ID NO: 1. In some embodiments, the DNA sequence encoding for O-GlycNAC transferase (OGT) can be SEQ ID NO: 1.

In some embodiments, the O-GlycNAC transferase, a fragment, or a variant thereof comprises an amino acid sequence with at least 80%, at least 85%, at least 90%, at least 95%, at least 96%, at least 97%, at least 98%, at least 99% sequence identity to SEQ ID NO: 2. In some embodiments, the O-GlycNAC transferase, a fragment, or a variant thereof comprises an amino acid sequence with at least 80% sequence identity to SEQ ID NO: 2. In some embodiments, the O-GlycNAC transferase, a fragment, or a variant thereof comprises an amino acid sequence with at least 90% sequence identity to SEQ ID NO: 2. In some embodiments, the O-GlycNAC transferase, a fragment, or a variant thereof comprises an amino acid sequence with at least 95% sequence identity to SEQ ID NO: 2. In some embodiments, the DNA encoding O-GlycNAC transferase (OGT) is circular. In some embodiments, the mRNA encoding O-GlycNAC transferase (OGT) is circular.

### Methods of Administration

The O-glycnacylation modifier agent, an inflammation agent inducer, and/or angiogenic factor as used in the methods described herein can be administered by any suitable method and technique presently or prospectively known to those skilled in the art. For example, the O-glycnacylation modifier agent, an inflammation agent inducer, and/or angiogenic factor described herein can be formulated in a physiologically- or pharmaceutically-acceptable form and administered by any suitable route known in the art including, for example, topical (as by powders, ointments, creams, and/or drops), aerosol, intravenous, subcutaneous, transcutaneous, transdermal, intramuscular, intradermal, intra-arteriole, intralesional, or any combination thereof. In general, the most appropriate route of administration will depend upon a variety of factors including the nature of the O-glycnacylation modifier agent, an inflammation agent inducer, and/or angiogenic factor, the condition of the subject, etc.

The O-glycnacylation modifier agent, an inflammation agent inducer, and/or angiogenic factor may be administered in such amounts, time, and route deemed necessary in order to achieve the desired result. The exact amount of the O-glycnacylation modifier agent, an inflammation agent inducer, and/or angiogenic factor will vary from subject to subject, depending on the species, age, and general condition of the subject, the particular O-glycnacylation modifier agent, an inflammation agent inducer, and/or angiogenic factor, its mode of administration, its mode of activity, and the like.

The exact amount of an O-glycnacylation modifier agent, inflammation agent inducer, and/or angiogenic factor required to achieve a therapeutically effective amount will vary from subject to subject, depending on species, age, and general condition of a subject, severity of the side effects or disorder, identity of the particular compound(s), mode of administration, and the like. The amount to be administered to, for example, a child or an adolescent can be determined by a medical practitioner or person skilled in the art and can be lower or the same as that administered to an adult.

Useful dosages of the O-glycnacylation modifier agent, inflammation agent inducer, and/or angiogenic factor and compositions disclosed herein can be determined by comparing their in vitro activity, and in vivo activity in animal models. Methods for the extrapolation of effective dosages in mice, and other animals, to humans are known to the art.

The dosage ranges for the administration of the O-glycnacylation modifier agent, inflammation agent inducer, and/or angiogenic factor are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Administration of the O-glycnacylation modifier agent, an inflammation agent inducer, and an angiogenic factor can be a single administration, or at continuous and distinct intervals as can be readily determined by a person skilled in the art. It will be understood, that the total daily usage of the O-glycnacylation modifier agent, inflammation agent inducer, and/or angiogenic factor will be decided by the attending physician within the scope of sound medical judgment. The specific therapeutically effective dose level for any particular subject will depend upon a variety of factors including the disorder being treated and the severity of the disorder: the activity of the active ingredient employed; the specific composition employed; the age, body weight, general health, sex and diet of the patient; the time of administration, route of administration, and rate of excretion of the specific active ingredient employed; the duration of the treatment; drugs used in combination or coincidental with the specific active ingredient employed; and like factors well known in the medical arts.

The O-glycnacylation modifier agent, an inflammation agent inducer, and/or an angiogenic factor described herein can be formulated to include an excipient of some sort. “Excipients” include any and all solvents, diluents or other liquid vehicles, dispersion or suspension aids, surface active agents, isotonic agents, thickening or emulsifying agents, preservatives, solid binders, lubricants, natural oils and the like, as suited to the particular dosage form desired. General considerations in formulation and/or manufacture can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

Exemplary excipients include, but are not limited to, any non-toxic, inert solid, semisolid or liquid filler, diluent, encapsulating material or formulation auxiliary of any type. Some examples of materials which can serve as excipients include, but are not limited to, sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soy bean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. As would be appreciated by one of skill in this art, the excipients may be chosen based on what the composition is useful for. For example, with a pharmaceutical composition, the choice of the excipient will depend on the route of administration, the agent being delivered, time course of delivery of the agent, etc., and can be administered to humans and/or to animals, topically (as by powders, creams, ointments, or drops).

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxy vinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof. Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, and other preservatives.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxy benzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid. Other preservatives include tocopherol, tocopherol acetate, deteroxime mesylate, cetrimide, butylated hydroxyanisol (BHA), butylated hydroxytoluene (BHT), ethylenediamine, sodium lauryl sulfate (SLS), sodium lauryl ether sulfate (SLES), sodium bisulfite, sodium metabisulfite, potassium sulfite, potassium metabisulfite, Glydant Plus, Phenonip, methylparaben, Germall 115, Germaben II, Neolone, Kathon, and Euxyl. In certain embodiments, the preservative is an anti-oxidant. In other embodiments, the preservative is a chelating agent.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, etc., and combinations thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, etc., and combinations thereof.

Exemplary natural oils include almond, apricot kernel, avocado, babassu, bergamot, black current seed, borage, cade, chamomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadow foam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soy bean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and combinations thereof.

Liquid compositions include emulsions, microemulsions, solutions, suspensions, syrups, and elixirs. In addition to the active compound, the liquid composition may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable compositions, for example, injectable aqueous or oleaginous suspensions may be formulated according to the known art using suitable dispersing or wetting agents and suspending agents. The sterile injectable preparation may also be an injectable solution, suspension, or emulsion in a nontoxic parenterally acceptable diluent or solvent, for example, as a solution in 1,3-butanediol. Among the acceptable vehicles and solvents for pharmaceutical or cosmetic compositions that may be employed are water, Ringer's solution, U.S.P. and isotonic sodium chloride solution. In addition, sterile, fixed oils are conventionally employed as a solvent or suspending medium. Any bland fixed oil can be employed including synthetic mono- or diglycerides. In addition, fatty acids such as oleic acid are used in the preparation of injectables. In certain embodiments, the particles are suspended in a carrier fluid comprising 1% (w/v) sodium carboxymethyl cellulose and 0.1% (v/v) Tween 80. The injectable composition can be sterilized, for example, by filtration through a bacteria-retaining filter, or by incorporating sterilizing agents in the form of sterile solid compositions which can be dissolved or dispersed in sterile water or other sterile injectable medium prior to use.

Solid compositions include capsules, tablets, pills, powders, and granules. In such solid compositions, the particles are mixed with at least one excipient and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets, and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

Compositions for topical or transdermal administration include ointments, pastes, creams, lotions, gels, powders, solutions, sprays, inhalants, or patches. The active compound is admixed with an excipient and any needed preservatives or buffers as may be required.

The ointments, pastes, creams, and gels may contain, in addition to the active compound, excipients such as animal and vegetable fats, oils, waxes, paraffins, starch, tragacanth, cellulose derivatives, polyethylene glycols, silicones, bentonites, silicic acid, talc, and zinc oxide, or mixtures thereof.

Powders and sprays can contain, in addition to the active compound, excipients such as lactose, talc, silicic acid, aluminum hydroxide, calcium silicates, and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the nanoparticles in a proper medium. Absorption enhancers can also be used to increase the flux of the compound across the skin. The rate can be controlled by either providing a rate controlling membrane or by dispersing the particles in a polymer matrix or gel.

The O-glycnacylation modifier agent, inflammation agent inducer, and/or angiogenic factor described herein can be incorporated into injectable/implantable solid or semi-solid implants, such as polymeric implants. In one embodiment, the compounds are incorporated into a polymer that is a liquid or paste at room temperature, but upon contact with aqueous medium, such as physiological fluids, exhibits an increase in viscosity to form a semi-solid or solid material. Exemplary polymers include, but are not limited to, hydroxyalkanoic acid polyesters derived from the copolymerization of at least one unsaturated hydroxy fatty acid copolymerized with hydroxyalkanoic acids. The polymer can be melted, mixed with the active substance and cast or injection molded into a device. Such melt fabrication requires polymers having a melting point that is below the temperature at which the substance to be delivered and polymer degrade or become reactive. The device can also be prepared by solvent casting where the polymer is dissolved in a solvent and the drug dissolved or dispersed in the polymer solution and the solvent is then evaporated. Solvent processes require that the polymer be soluble in organic solvents. Another method is compression molding of a mixed powder of the polymer and the drug or polymer particles loaded with the O-glycnacylation modifier agent.

Alternatively, the O-glycnacylation modifier agent, inflammation agent inducer, and/or angiogenic factor can be incorporated into a polymer matrix and molded, compressed, or extruded into a device that is a solid at room temperature. For example, the compounds can be incorporated into a biodegradable polymer, such as polyanhydrides, polyhydroalkanoic acids (PHAs), PLA, PGA, PLGA, polycaprolactone, polyesters, polyamides, polyorthoesters, polyphosphazenes, proteins and polysaccharides such as collagen, hyaluronic acid, albumin and gelatin, and combinations thereof and compressed into solid device, such as disks, wafers, or extruded into a device, such as rods.

The release of the O-glycnacylation modifier agent, inflammation agent inducer, and/or angiogenic factor from the implant can be varied by selection of the polymer, the molecular weight of the polymer, and/or modification of the polymer to increase degradation, such as the formation of pores and/or incorporation of hydrolyzable linkages. Methods for modifying the properties of biodegradable polymers to vary the release profile of the compounds from the implant are well known in the art.

In some embodiments, the O-glycnacylation modifier agent, inflammation agent inducer, and/or angiogenic factor can be administered locally. In some embodiments, the O-glycnacylation modifier agent, inflammation agent inducer, and/or angiogenic factor are incorporated in a delivery system such as gels, nanoparticles, microparticles, or implants such as (e.g., rods, discs, wafers, orthopedic implants) for sustained release.

The O-glycnacylation modifier agent, inflammation agent inducer, and/or angiogenic factor can be incorporated microparticles, nanoparticles, or combinations thereof that provide controlled release of the agents. For example, the agents can be incorporated into polymeric microparticles, which provide controlled release of the drug(s). Release of the agents is controlled by diffusion of the agents out of the microparticles and/or degradation of the polymeric particles by hydrolysis and/or enzymatic degradation. Suitable polymers include ethylcellulose and other natural or synthetic cellulose derivatives.

Polymers, which are slowly soluble and form a gel in an aqueous environment, such as hydroxypropyl methylcellulose or polyethylene oxide, may also be suitable as materials for microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxy butyrate (PHB) and copolymers thereof, poly-4-hydroxy butyrate (P4HB) and copolymers thereof, polycaprolactone and copolymers thereof, and combinations thereof.

Alternatively, the O-glycnacylation modifier agent, inflammation agent inducer, and/or angiogenic factor can be incorporated into microparticles prepared from materials which are insoluble in aqueous solution or slowly soluble in aqueous solution, but are capable of degrading by means including enzymatic degradation, and/or mechanical erosion. As used herein, the term “slowly soluble in water” refers to materials that are not dissolved in water within a period of 30 minutes. Preferred examples include fats, fatty substances, waxes, wax-like substances and mixtures thereof. Suitable fats and fatty substances include fatty alcohols (such as lauryl, myristyl stearyl, cetyl or cetostearyl alcohol), fatty acids and derivatives, including but not limited to fatty acid esters, fatty acid glycerides (mono-, di- and tri-glycerides), and hydrogenated fats. Specific examples include, but are not limited to hydrogenated vegetable oil, hydrogenated cottonseed oil, hydrogenated castor oil, hydrogenated oils available under the trade name Sterotex®, stearic acid, cocoa butter, and stearyl alcohol. Suitable waxes and wax-like materials include natural or synthetic waxes, hydrocarbons, and normal waxes. Specific examples of waxes include beeswax, glycowax, castor wax, carnauba wax, paraffins and candelilla wax. As used herein, a wax-like material is defined as any material, which is normally solid at room temperature and has a melting point of from about 30 to 300° C.

In some cases, it may be desirable to alter the rate of water penetration into the microparticles. To this end, rate-controlling (wicking) agents may be formulated along with the fats or waxes listed above. Examples of rate-controlling materials include certain starch derivatives (e.g., waxy maltodextrin and drum dried corn starch), cellulose derivatives (e.g., hydroxypropylmethyl-cellulose, hydroxy propylcellulose, methylcellulose, and carboxymethyl-cellulose), alginic acid, lactose and talc. Additionally, a pharmaceutically acceptable surfactant (for example, lecithin) may be added to facilitate the degradation of such microparticles.

Proteins, which are water insoluble, such as zein, can also be used as materials for the formation of microparticles containing O-glycnacylation modifier agent, inflammation agent inducer, and/or angiogenic factor.

Encapsulation or incorporation of O-glycnacylation modifier agent, inflammation agent inducer, and/or angiogenic factor into carrier materials to produce drug-containing microparticles can be achieved through known pharmaceutical formulation techniques. In the case of formulation in fats, waves or wax-like materials, the carrier material is typically heated above its melting temperature and the drug is added to form a mixture comprising drug particles suspended in the carrier material, drug dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, drug is added, and the molten wax-drug mixture is congealed under constant stirring as the mixture cools. Alternatively, the molten wax-drug mixture can be extruded and spheronized to form pellets or beads. These processes are known in the art.

For some carrier materials it may be desirable to use a solvent evaporation technique to produce drug-containing microparticles. In this case drug and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, drug(s) in a particulate form is homogeneously dispersed in a water-insoluble or slowly water-soluble material. To minimize the size of the drug particles within the composition, the drug powder itself may be milled to generate fine particles prior to formulation. The process of jet milling, known in the pharmaceutical art, can be used for this purpose. In some embodiments, drug in a particulate form is homogeneously dispersed in a wax or wax like substance by heating the wax or wax like substance above its melting point and adding the drug particles while stirring the mixture. In this case a pharmaceutically acceptable surfactant may be added to the mixture to facilitate the dispersion of the drug particles.

The particles can also be coated with one or more modified release coatings. Solid esters of fatty acids, which are hydrolyzed by lipases, can be spray coated onto microparticles or drug particles. Zein is an example of a naturally water-insoluble protein. It can be coated onto drug containing microparticles or drug particles by spray coating or by wet granulation techniques. In addition to naturally water-insoluble materials, some substrates of digestive enzymes can be treated with cross-linking procedures, resulting in the formation of non-soluble networks. Many methods of cross-linking proteins, initiated by both chemical and physical means, have been reported. One of the most common methods to obtain cross-linking is the use of chemical cross-linking agents. Examples of chemical cross-linking agents include aldehydes (gluteraldehyde and formaldehyde), epoxy compounds, carbodiimides, and genipin. In addition to these cross-linking agents, oxidized and native sugars have been used to cross-link gelatin. Cross-linking can also be accomplished using enzymatic means; for example, transglutaminase has been approved as a GRAS substance for cross-linking seafood products. Finally, cross-linking can be initiated by physical means such as thermal treatment, UV irradiation and gamma irradiation.

To produce a coating layer of cross-linked protein surrounding drug containing microparticles or drug particles, a water-soluble protein can be spray coated onto the microparticles and subsequently cross-linked by the one of the methods described above. Alternatively, drug-containing microparticles can be microencapsulated within protein by coacervation-phase separation (for example, by the addition of salts) and subsequently cross-linked. Some suitable proteins for this purpose include gelatin, albumin, casein, and gluten.

Polysaccharides can also be cross-linked to form a water-insoluble network. For many polysaccharides, this can be accomplished by reaction with calcium salts or multivalent cations, which cross-link the main polymer chains. Pectin, alginate, dextran, amylose and guar gum are subject to cross-linking in the presence of multivalent cations. Complexes between oppositely charged polysaccharides can also be formed; pectin and chitosan, for example, can be complexed via electrostatic interactions.

In certain embodiments, it may be desirable to provide continuous delivery. For intravenous or intraarterial routes, this can be accomplished using drip systems, such as by intravenous administration. For topical applications, repeated application can be done or a patch can be used to provide continuous administration of the compounds over an extended period of time.

### EXAMPLES

### Example 1

Transdifferentiation from fibroblasts to endothelial cells contribute to the angiogenic response in the recovery of hindlimb ischemia. A glycolytic switch is required during this process. Hexosamine biosynthesis pathway is activated in glycolysis to produce UDP-GlcNAc for protein O-GlcNacylation. A single pair of enzymes-O-GlcNAc transferase (OGT) and O-GlcNAcase (OGA)-controls the dynamic cycling of this protein modification. O-GlcNAcylation may be involved in transdifferentiation and hindlimb ischemia recovery.

Using established transdifferentiation protocol and metabolomics profiling to assess the global metabolic changes during transdifferentiation. It was observed that UDP-GlcNAc was the most upregulated metabolite during transdifferentiation. To determine the role of this metabolite in transdifferentiation, the effect of Thiamet G was assessed, an inhibitor of OGA, which enhances O-GlcNAcylation. Thiamet G increased transdifferentiation efficiency in vitro. In a fibroblast lineage tracing mice, Fsp1-Cre: R26R-EYFP mice, Thiamet G increased tissue O-glycnacylation, increased capillary density and limb perfusion, and increased the number of fibroblasts transdifferentiating into endothelial cells (i.e. FSP+CD31+ cell) in an ischemic hindlimb model. Finally, in Col1a2-Cre/ERT:OGT flox/flox mice (knockout of OGT in Col1a2 expressing fibroblasts induced by tamoxifen), exhibited impaired restoration of perfusion after femoral artery ligation.

O-GlcNAcylation is upregulated during ischemia; enhances transdifferentiation of fibroblasts to endothelial cells; and augments the recovery from hindlimb ischemia. These data shed light on a new angiogenic process mediated by O-GlcNAcylation.

### INTRODUCTION

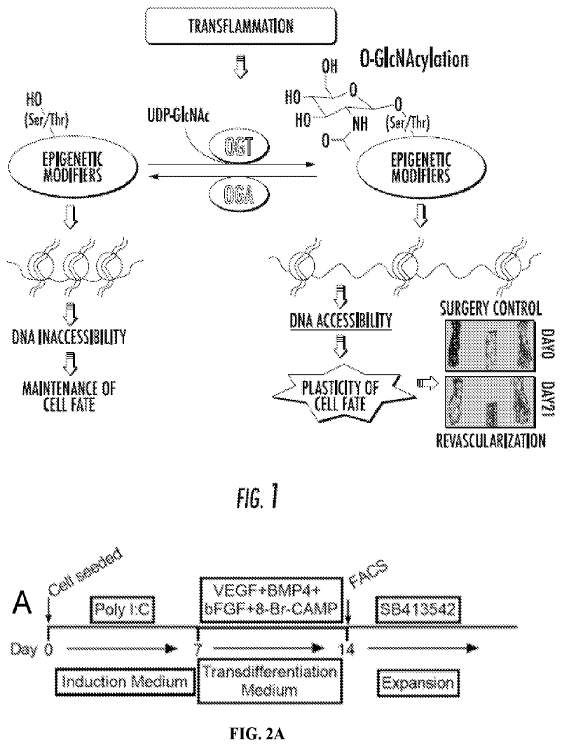

O-GlcNAcylation has been shown to modify the activity of epigenetic modifiers, the following steps were taken to test if O-GlcNAcylation facilitates cell fate plasticity and enhances limb ischemia recovery (FIG. 1).

Determine the mechanism of epigenetic regulation of O-GlcNAcylation in DNA accessibility during transdifferentiation. A. To determine if O-GlcNAcylation is required for transdifferentiation, O-GlcNAcylation can be altered genetically or pharmacologically during in vitro transdifferentiation. B. O-GlcNAc proteomics can be employed to identify O-GlcNAcylation sites on epigenetic modifiers during transdifferentiation. C. The effect of O-GlcNAcylation on DNA accessibility can be studied with single-cell multiomics (scATAC seq and scRNA seq), by inducing transdifferentiation in the O-GlcNAcylation genetically modified fibroblasts.

Determine the significance of O-GlcNAcylation in transdifferentiation during limb ischemia recovery. A. To determine if the elevation of O-GlcNAcylation occurs in transdifferentiation during recovery, a hindlimb ischemia model can be used in lineage-tracing mice, assessing the O-GlcNAcylation pathway in the fibroblast subsets in the ischemic limb. B. To identify if manipulation of O-GlcNAcylation in fibroblasts affects transdifferentiation in vivo and limb ischemia recovery, fibroblast-specific KO mice for OGT/OGA can be studied, the pair of enzymes responsible for adding or removing this modification respectively.

Background: The studies demonstrate a mechanism for harnessing an endogenous mechanism that expands the microcirculation by the trasdifferentiation of fibroblasts to endothelial cells. These studies can link metabolic changes to cell fate transitions that can be generally relevant to most cardiovascular diseases. Finally, identification of this pathway can provide novel targets not only for limb ischemia and regeneration but potentially for the prevention of this process associated with metabolic disorders. The potential for metabolic control of epigenetic reprogramming and angiogenesis/endothelial cell differentiation. Targeted metabolic approach, combined with novel global approaches using contemporary tools in MS-based O-GlcNAc proteomics (O-GlcNAcomics), multiomics, bioinformatics, and molecular biology can be used.

Angiogenic transdifferentiation and inflammatory signaling. The group determined that transflammation enhances cell fate plasticity by downregulating histone deacetylase (HDACs); up-regulating histone acetyltransferases (HATs); and reducing the activity of suppressive factors (Chanda, P. K., et al., Circulation, 2019. 140 (13): p. 1081-1099; Meng, S., et al., Circ Res, 2016. 119 (9): p. e129-e138). Its activation is required for nuclear reprogramming to pluripotency (Lee, J., et al., Cell, 2012. 151 (3): p. 547-58) and transdifferentiation from fibroblast to endothelial cells) (Sayed, N., et al., Circulation, 2015. 131 (3): p. 300-9). In recent studies, a subset of tissue fibroblasts undergoes angiogenic transdifferentiation to endothelial cells in an ischemic hindlimb model and contribute to the recovery of perfusion (Meng, S., et al., Circulation, 2020. 142 (17): p. 1647-1662). Therapeutic modulation to expand this fibroblast subset may represent a novel strategy for enhancing vascular regeneration in ischemic diseases.

Interplay between metabolism and epigenetics in cardiovascular diseases. Epigenetic programming via chromatin-modifying enzymes and the metabolic regulation of DNA accessibility has emerged as a key regulatory process controlling cell fate (Li, X., et al., Nat Rev Mol Cell Biol, 2018. 19 (9): p. 563-578). The studies identified a glycolytic switch, in which glycolysis is preferred even in an aerobic environment, and essential for histone acetylation, epigenetic modification, and transflammation (Lai, L., et al., Circulation, 2019. 139 (1): p. 119-133). Surprisingly, global metabolic profiling of this in vitro transdifferentiation suggests an upregulation of UDP-GlcNAc and O-GlcNAcylation. O-GlcNAcylation is an essential post-translational protein modification, which often directly alters their activity (Bond, M. R., eta al., J Cell Biol, 2015. 208 (7): p. 869-80). O-GlcNAc-transferase (OGT) and O-GlcNAcase (OGA) are the pair of enzymes that add or remove this protein modification respectively. Accumulating data have revealed that O-GlcNAcylation is essential in the modulation of chromatin remodeling by modifying histone tails and epigenetic modifiers (Leturcq, M., et al., Biochem Soc Trans, 2017. 45 (2): p. 323-338; Sakabe, K., et al., Proc Natl Acad Sci USA, 2010. 107 (46): p. 19915-20). Studies showed O-GlcNAcylation confers protection following acute ischemic reperfusion and other types of cardiovascular injuries by improving post-ischemic contractile function recovery in the heart (Liu, J., et al., J Mol Cell Cardiol, 2006. 40 (2): p. 303-12). However, its role in limb ischemia recovery and transdifferentiation in the ischemic syndromes requires further investigation.

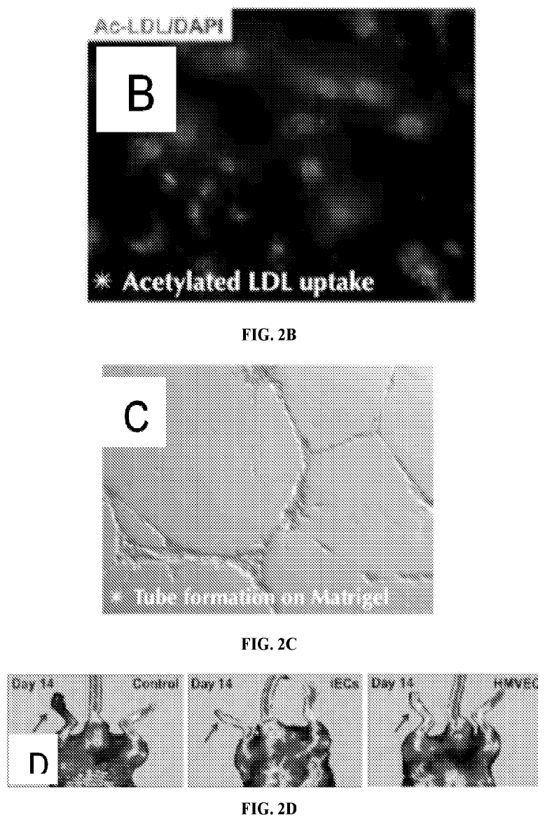

Innate immune activation is required for transdifferentiation. The lab has previously established a protocol for transdifferentiate fibroblasts into induced endothelial cells (iECs) (FIG. 2A), which utilizes TLR3 agonist Polyinosinic:polycytidylic acid (PolyI:C) to induce inflammatory signaling, together with endothelial growth factors including VEFG, FGF, BMP4, and 8-Br-CAMP, to provide transcriptional direction (note: both inflammatory signaling and transcriptional cues are required for transdifferentiation of fibroblasts to endothelial cells; transdifferentiation does not occur with PolyI:C or endothelial growth factors alone) (Sayed, N., et al., Circulation, 2015. 131 (3): p. 300-9). iECs generated from this protocol manifest high fidelity for endothelial lineage. They incorporate acetylated LDL (FIG. 2B), form tubular networks (FIG. 2C), have similar transcriptomes as genuine endothelial cells, and increase capillary density and perfusion when injected into the ischemic hindlimb of the mouse (FIG. 2D) (Sayed, N., et al., Circulation, 2015. 131 (3): p. 300-9). This protocol can be utilized to study the role of O-GlcNAcylation in transdifferentiation in vitro.

Angiogenic transdifferentiation in limb ischemia. A fibroblast lineage tracing mouse model (Fsp1-Cre: R26R-EYFP) was used to detect transdifferentiation in vivo in a murine model of limb ischemia (Meng, S., et al., Circulation, 2020. 142 (17): p. 1647-1662). The results showed that YFP+CD11b-fibroblasts transdifferentiated into endothelial cells and accounted for 4% to 6% of the total CD144+endothelial cells at day 21 after the induction of ischemia. This population is abolished when the mice are treated with anti-inflammatory drugs, such as dexamethasone. Intriguingly, the disappearance of these cells is associated with a reduction in perfusion recovery and worsening clinical scores. Similar results are achieved when inflammatory signaling is impaired, as in Fsp1-Cre: Rela flox/flox mice. In these animals, NFκB signaling is blocked in the Fsp1+fibroblasts, associated with a dramatic reduction in transdifferentiated fibroblasts (i.e. YFP+CD144+ cells), and impaired perfusion (Meng. S., et al., Circulation, 2020. 142 (17): p. 1647-1662). Single-cell RNAseq studies of transdifferentiation revealed 8 discrete clusters of YFP+ cells with Clusters 5 and 8 expanding dramatically during recovery from ischemia. Cluster 8 produced angiogenic cytokines, whereas Cluster 5 expressed genes associated with EC identity (i.e CD144). But only Cluster 5 could generate EC-like networks in Matrigel whereas other fibroblasts, including Cluster 8 fibroblasts, formed nodal structures typical of fibroblasts in Matrigel. The data support the idea that transdifferentiation may occur in vivo with tissue ischemia, and is dependent on inflammatory signaling. This model can be used to study the contribution of O-GlcNAcylation in transdifferentiation and limb ischemia recovery in vivo. A similar lineage tracing approach can be used with tamoxifen-inducible Col1A2-Cre/ERT: R26R-tdTomato mouse strain to study the contribution of O-GlcNAcylation in transdifferentiation and limb ischemia recovery in vivo.

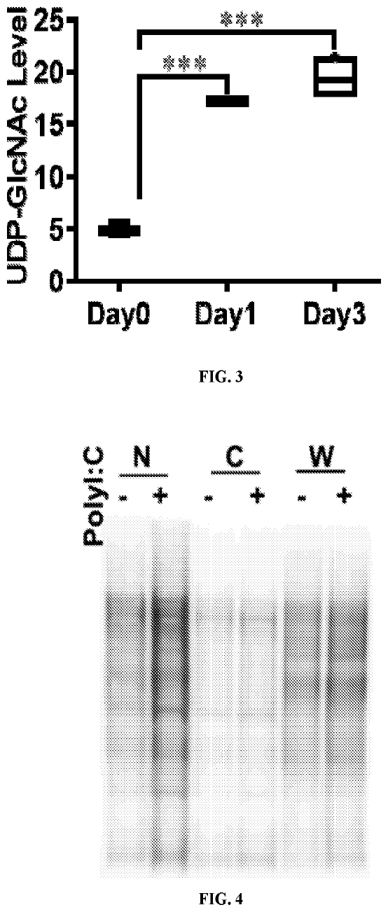

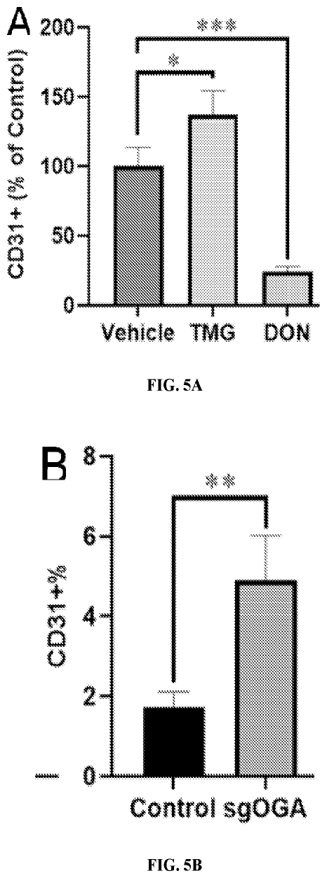

Cellular O-GlcNAcylation is elevated during transdifferentiation. To facilitate transdifferentiation of fibroblasts to endothelial cells in vitro, the TLR3 agonist Polyinosinic:polycytidilic acid (PolyIC) was used to induce inflammatory signaling, together with endothelial growth factors to provide transcriptional direction (both inflammatory signaling and transcriptional cues are required for; transdifferentiation of fibroblasts to endothelial cells; transdifferentiation does not occur with PolyIC or endothelial growth factors alone). TLR3 activation increases DNA accessibility and facilitates transdifferentiation. Other forms of inflammatory activation also increase DNA accessibility (as with RIG1 activation). LCMS-based metabolomic analysis of the fibroblasts that underwent transdifferentiation was used to determine that UDP-GlcNAc is highly elevated PolyI:C activation of fibroblasts (FIG. 3). Principal Component Analysis (PCA) shows clear separation in different time point groups and consistency among the triplicates of each time point. To determine if the increase in UDP-GlcNAc is associated with an increase in O-GlcNAcylation, cells were treated with PolyI:C or vehicle for 3 days, then the O-GlcNAcylated proteins in the nuclear (N), cytoplasmic (C), or whole-cell (W) lysates were enzymatically labeled with azido-modified galactose (GalNAz), which were further detected by click-it chemistry using a biotinylated alkyne and Western. The results comparing the PolyI:C treated and untreated groups show that O-GlcNAcylation elevation primarily occurs in nuclear proteins (FIG. 4).

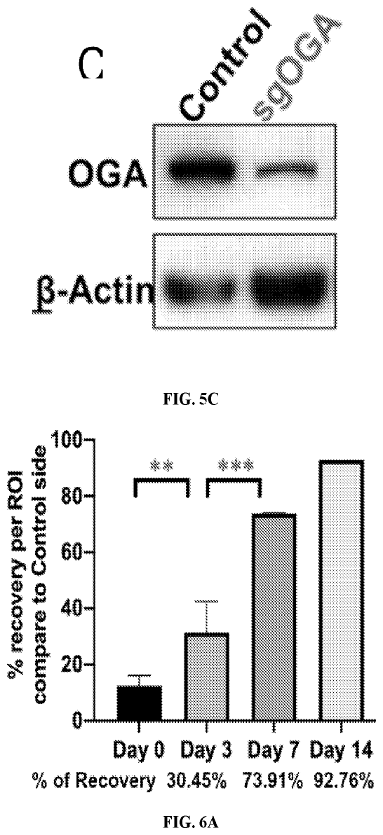

O-GlcNAcylation is required for in vitro transdifferentiation. To define if the elevation of O-GlcNAcylation is required for transdifferentiation, fibroblasts were exposed to Thiamet G (TMG) (an OGA inhibitor that enhances O-GlcNAcylation) or DON (an inhibitor for GFAT that acts upstream in the hexosamine biosynthesis pathway and reduces O-GlcNAcylation). The number of iECs generated during transdifferentiation is increased when fibroblasts are exposed to TMG and decreased when exposed to DON (FIG. 5A). A stable knockdown of OGA in the fibroblasts, generated by the CRISPR-cas9 system to enhance O-GlcNAcylation (FIG. 5C), revealed that transdifferentiation was enhanced by blocking OGA to increase O-GlcNAcylation (FIG. 5B). These data suggest that transdifferentiation is regulated by O-GlcNAcylation. The role of O-GlcNAcylation of specific epigenetic regulars e.g., HIRA during transdifferentiation can also be addressed.

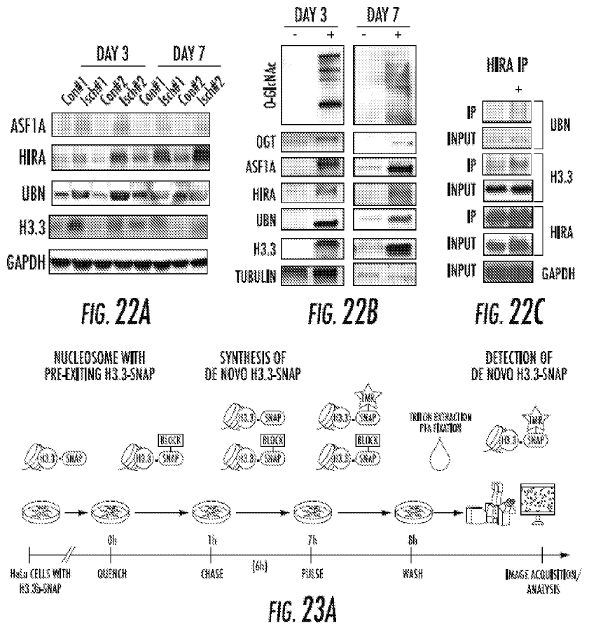

DNA accessibility is enhanced during transdifferentiation. DNA accessibility is critical for the epigenetic regulation of cell fate transition. Transcriptional and chromatin accessibility profiling data shows that inflammatory activation during transdifferentiation upregulates more genes than it down-regulates (FIG. 25A). Signal enrichment around transcriptional start sites (TSSs) shows that cells undergoing transdifferentiation have more accessible chromatin regions (FIG. 25B). ATAC seq can be used to determine how modulation of O-GlcNAcylation affects DNA accessibility.

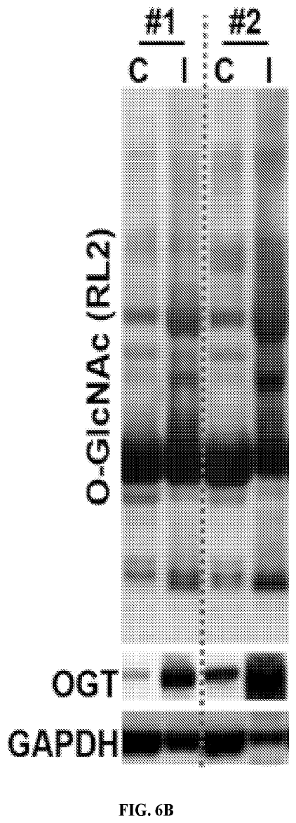

O-GlcNAcylation is increased during recovery from ischemia. Using the mouse limb ischemia recovery model in WT C57BL/6 mice, perfusion was assessed by Doppler flow imaging (FIG. 6A), and changes in O-GlcNAcylation were quantified at different time intervals over the 14-day post-surgery recovery. Intriguingly, Western blotting (FIG. 6B) of hindlimb tissues revealed a substantial increase in O-GlcNAcylation and OGT expression in the ischemic limb in both animals (#1 and #2) at day 3 post-surgery. Similar findings were observed at day 7 but had resolved by day 14, at which time perfusion had substantially recovered (FIG. 6A). Altogether, this evidence suggests that O-GlcNAcylation is increased with tissue ischemia and resolves during recovery. The role of O-GlcNAcylation-dependent epigenetic regulation during ischemia will be determined.

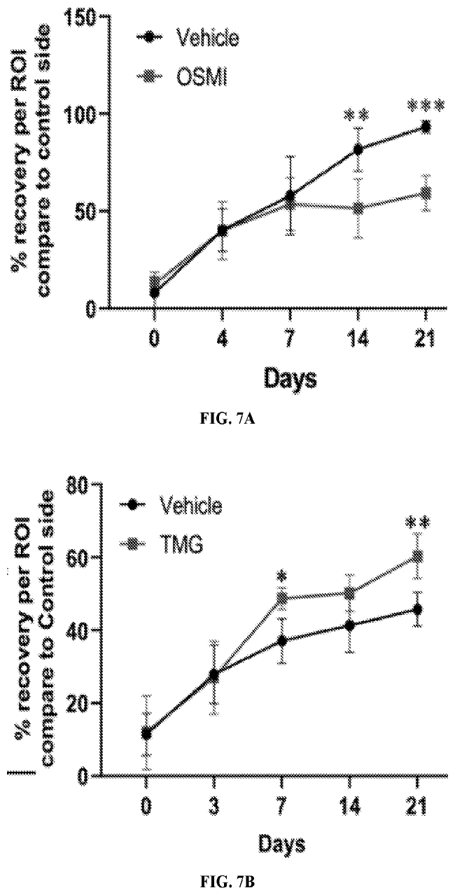

O-GlcNAcylation manipulations regulated recovery from tissue ischemia. To determine if the change in O-GlcNAcylation can modulate limb ischemia recovery, and contributes to vascular recovery, WT C57BL/6 mice were treated with the O-GlcNAcylation inhibitor, OSMI4, or O-GlcNAcylation enhancer, TMG 0, 1, 2 days post-surgery. Age is an important factor that determines recovery, and younger mice are known to have a more robust angiogenic response as compared with older mice. Accordingly, younger mice (between 8 to 12 weeks) were used to assess the impairment of angiogenesis by OSMI (which reduces O-GlcNAcylation). Older mice (around 20 weeks) were used to assess the enhancement of angiogenesis by TMG (which increases O-GlcNAcylation). OSMI impaired recovery, while TMG enhanced recovery of blood flow post-femoral artery ligation (FIG. 7A-7B). These data suggest that O-GlcNAcylation regulates vascular regeneration during the recovery from limb ischemia. It will be determined whether this beneficial effect is mediated by increased DNA accessibility and enhanced transdifferentiation.

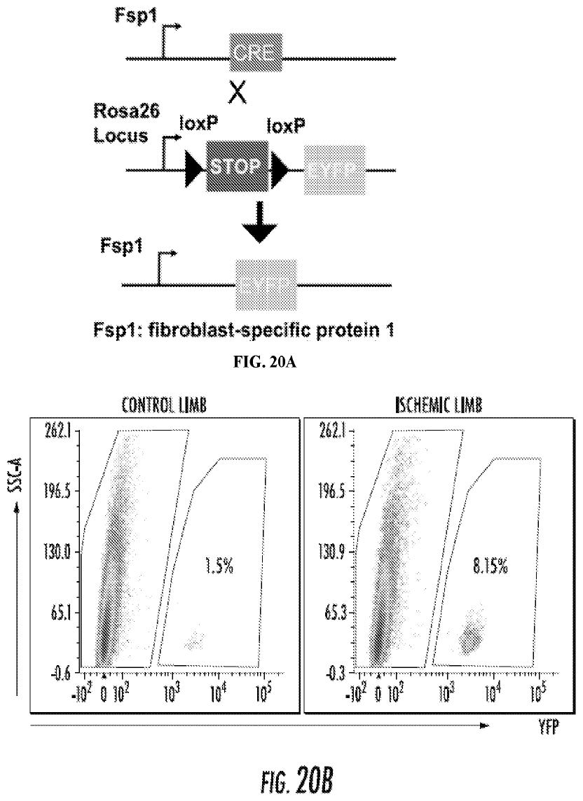

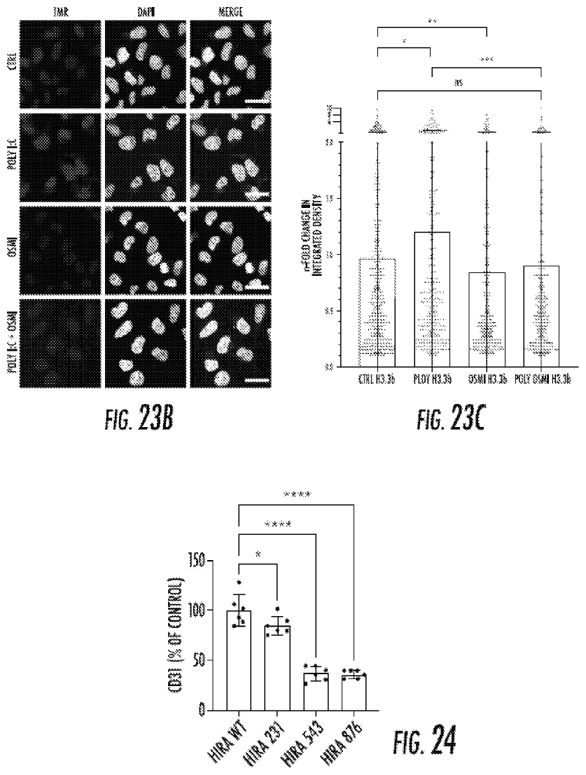

O-GlcNAcylation level increases in the fibroblast lineage cells during vascular recovery. To study the role of O-GlcNAcylation in transdifferentiation in vivo, the lineage tracing model was used, Fsp1-Cre: R26R-EYFP where fibroblasts expressing Fsp1 (fibroblast-specific protein1) are marked with YFP and are CD11b−. An increase in the Fspl-expressing cells in the ischemic limb 3 days post femoral artery ligation was observed (FIG. 20B) suggesting the fibroblasts are rapidly activated in response to the ischemia. Intriguingly, when sorted from the disaggregated limb tissue 3 days post-surgery, these YFP. CD11b-fibroblasts manifest a marked increase in O-GlcNAcylation by Western analysis (FIG. 26). Furthermore, the level of histone protein 3 variant h3.3, which usually marks activated transcription, and the subunits of the h3.3 chaperone HIRA complex 26, including HIRA, ASF1, and UBN are all significantly accumulated in the YFP+ population, especially in the ischemic limb, in a trend similar to that of O-GlcNAcylation. These data further suggest a dramatic metabolic and epigenetic change in the YFP+ population and its potentially important functional role during vascular recovery. A tamoxifen-inducible Col1A2-Cre/ERT: R26R-tdTomato can be used to further elucidate the role of O-GlcNAcylation on epigenetic determinants of DNA accessibility and transdifferentiation during revascularization.

### Methods

Male or female healthy mice, of approximately 6 weeks to 5 months of age are used but sexes will remain consistent per experiment. For all quantitative studies, n=20 mice per group were used. Power analysis based on experiments indicates that such sample size provides approximately 80% power and alpha 0.05 to detect a difference. All results are shown as mean-value along with ±SEM of 2 to 5 biological replicates. For comparison between two groups, P values are determined using the Student t-test. For multiple comparisons, 2-way ANOVA is used, with P value further corrected by using Turkey's method (*, 0.01<P<0.05; **, 0.001<P<0.01; ***, P<0.001).

### Determine the Mechanism of Epigenetic Regulation of O-GlcNAcylation in DNA Accessibility During Transdifferentiation.

Data indicates that O-GlcNAcylation is required for transdifferentiation in vitro. However, it remains unclear if O-GlcNAcylation is a driver of this process and how this metabolic regulation affects epigenetic regulation and DNA accessibility, which are key processes for cell fate conversion. In vitro transdifferentiation protocol and characterization of endothelial functions has been established. Collaboration for O-GlcNAc proteomics and multi-omics analysis was also established for the determination of the downstream epigenetic effects of this metabolic change. Studies can be used to established in vitro transdifferentiation protocol to determine if altering O-GlyNAcylation alter transdifferentiation. O-GlcNAc proteomics and multi-omics analysis can be used to determine the downstream epigenetic effects of this metabolic change. DNA accessibility can be assessed with ATAC seq after modulating O-GlcNAcylation. Further, the effect of O-GlcNAcylation on one of its substrates, HIRA, and on histone variant h3.3 deposition during transdifferentiation can be studied.

### Experimental:

Assess the role of the O-GlcNAcylation during transdifferentiation. To further confirm the data that the addition of TMG and DON (FIG. 5A), and knockdown OGA (FIG. 5B) during transdifferentiation can alter this process additional pharmacological activators/inhibitors (PUGNAc (enhancer), OSMI (inhibitor)) can be added to fibroblasts including a subset that possess an OGT knockdown and production of iECs can be quantified by flow cytometry for CD31+CD144+iECs. These cells can also be isolated and their phenotype can be assessed by IF staining of CD31 and CD144 as well as functionally characterized to determine if they can undergo tube formation, migration, nitric oxide production, and acetylated LDL uptake similar to genuine ECs. Inhibition of O-GlcNAcylation can decrease transdifferentiation, while alternatively activation of this process should enhance iEC formation. However, it cannot be rule out that this in vitro system is maximally activated. Additionally, OGT knockdown may be lethal to cells. If so, then an inducible knockdown system can be used.

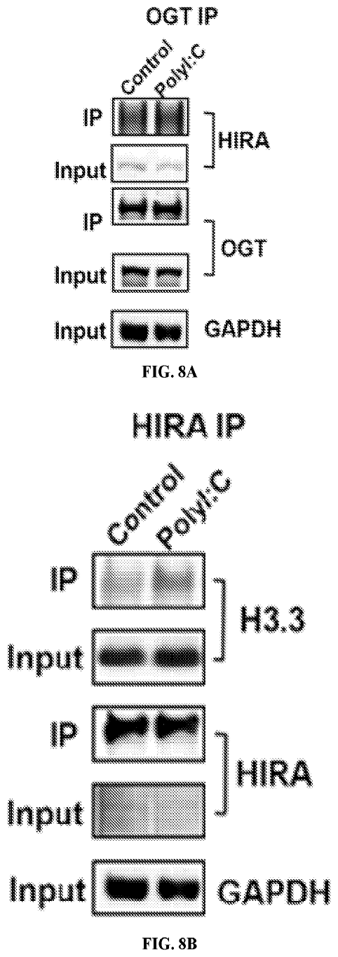

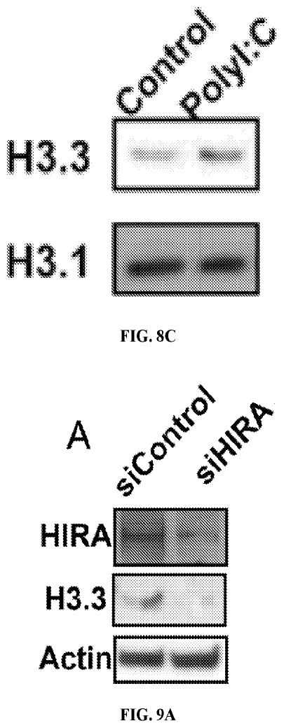

Identify the role of O-GlcNAcylation in epigenetic regulation of transdifferentiation. Use MS-based O-GlcNAc proteomics (O-GlcNAcomic) (Thompson, J. W., et al., Methods Enzymol, 2018. 598: p. 101-135: Thompson, J. W., et al., Biochemistry, 2018. 57 (27): p. 4010-4018; and Li, J., et al., ACS Chem Biol, 2019. 14 (1): p. 4-10) to globally identify key epigenetic factors that are O-GlcNAcylated followed by inflammatory signaling activation. A targeted approach to quantify changes in epigenetic modifiers, which are known to be O-GlcNAcylated, such as HIRA was taken (Wulff-Fuentes, E., et al., Sci Data, 2021. 8 (1): p. 25), a chaperon for histone protein H3.3, which is responsible for the incorporation of this histone variant into the chromatin of active genes (Saleh. R. N. M., et al., Mol Biol Rep, 2018. 45 (5): p. 1001-1011). OGT modifies HIRA and O-GlcNAcylation of this modifier is critical in the formation of the HIRA-H3.3 complex and H3.3 dependent gene activation. Preliminary data indicate that inflammatory activation enhances the binding of OGT to HIRA (FIG. 8A), HIRA to H3.3 (FIG. 8B), and eventually increases the protein level of H3.3. Knockdown of HIRA using siRNA (FIG. 9A) also impairs transdifferentiation (FIG. 9B). Those studies showed the importance of the HIRA-H3.3 pathway in transdifferentiation and suggested O-GlcNAcylation may regulate transdifferentiation through such mechanisms. To determine if alteration of HIRA O-GlcNAcylation can alter H3.3 deposition and transdifferentiation can be studied. OGT/OGA knockdown cells can be used to determine the role of O-GlcNAcylation in HIRA-H3.3 complex integrity by performing the immunoprecipitation experiment to assess the interaction between subunits of the HIRA complex, including H3.3, HIRA, UBN, CABIN, ASF. H3.3-SNAP tagging system and the quench-pause-chase method can also be used to determine if H3.3 deposition is enhanced by PolyI:C treatment and further regulated by OGT/OGA knockdown. Specific sites of HIRA that are O-GlcNAcylated by PolyI:C can be identified by O-GlcNAcomic. To further assess the functional role of the identified O-GlcNAcylated sites on HIRA, these sites can be mutated in fibroblasts and then iEC transdifferentiation efficiency can be compared with control cells.

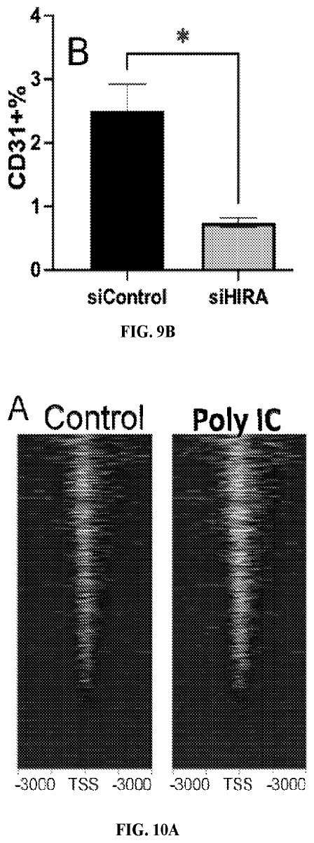

Identify the role of O-GlcNAcylation in DNA accessibility. Previous studies showed that PolyI:C treatment enhances DNA accessibility by micrococcal nuclease sensitivity assay in MEFs undergoing reprogramming to iPSCs (Chanda, P. K., et al., Circulation, 2019, 140 (13): p. 1081-1099). However, it is unknown if this occurs during transdifferentiation of fibroblasts to iECs. ATAC seq data analysis of signal enrichment around transcriptional start sites (TSSs) shows that PolyI:C treated cells have more accessible chromatin regions (FIG. 10A). RNA seq data also shows that there are more up-regulated genes than down-regulated genes (FIG. 10B). These data suggest that inflammatory signaling increases DNA accessibility. To test if O-GlcNAcylation controls this process.

OGT/OGA knockdown cells can be used to perform Multi-omics, which combines scATAC seq and scRNA seq on the same cells at the same time to qualitatively and quantitatively associate changes in chromatin accessibility with changes in gene expression. Computational biology can be used to analyze data and make comparisons of the accessible regions, motif enrichment/activity, and nucleosome positioning between control and OGT/OGA knockdown cells with or without PolyI:C treatment. From this analysis, key transcriptional factors downstream of the epigenetic modifiers can be identified and a comprehensive mechanism of transdifferentiation can be determined.

Results and alternative strategies: First, generate a list of epigenetic factors that are O-GlcNAcylated during activation of transflammation. Studies to block O-GlcNAcylation are expected to impair the HIRA-H3.3 complex integrity and H3.3 deposition, and the mutation of the HIRA O-GlcNAcylation site identified by O-GlcNAcomic in fibroblasts will impair transdifferentiation. There are other mechanisms for HIRA control of H3.3 function (Ray-Gallet, D., et al., Nat Commun. 2018. 9 (1): p. 3103; and Torne. J., et al., Nat Struct Mol Biol. 2020. 27 (11): p. 1057-1068), like HIRA homotrimer formation, that can be used if OGT/OGA knockout does not affect HIRA interaction with some of the other subunits. Moreover, the quench-pause-chase experiment with the H3.3-SNAP-tag system can determine if O-GlcNAcylation involves in H3.3 de novo deposition or retention which are through a different mechanism. Also, mutation of the HIRA O-GlcNAcylation sites may not be sufficient to affect transdifferentiation. Another subunit of the HIRA complex. UBN, is also known to be O-GlcNAcylated. If no changes are seen with HIRA mutation, the O-GlcNAcylation site on UBN can be used. Although HIRA is a candidate, more candidates and epigenetic-transcriptional networks can be identified through the global screen that is controlled by O-GlcNAcylation and is important for transdifferentiation by O-GlcNAcomics. These new candidates can also be addressed. Additionally. OGT/OGA can enhance/impair the innate immune-activated DNA accessibility, and a list of candidate TFs can be identified from multi-omics which can be further screened and confirmed in using in vitro and in vivo transdifferentiation models.

It is predicted that the OGT KD will impair, whereas OGA KD will increase the global DNA accessibility during transdifferentiation. Key downstream transcriptional phenomena mediated by O-GlcNAcylation manipulation during transdifferentiation can be identified. The epigenetic factors that are involved in transdifferentiation can also be identify. Those findings can be confirmed using in vitro and in vivo transdifferentiation models. Subpopulations and their relationships to one another can be identified, using single-cell RNAseq. and RNA velocity inference based on intron retention by varied methods including scVelo and cell Dancer (https://www.researchsquare.com/article/rs-1919313/v1), which is developed to estimate the direction and speed with which cells transition between clusters/states. OGT/OGA knockdown cells that undergo transdifferentiation can be used to confirm these changes are associated directly with O-GlcNAcylation.

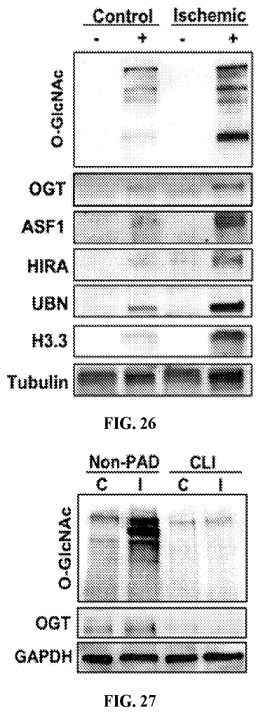

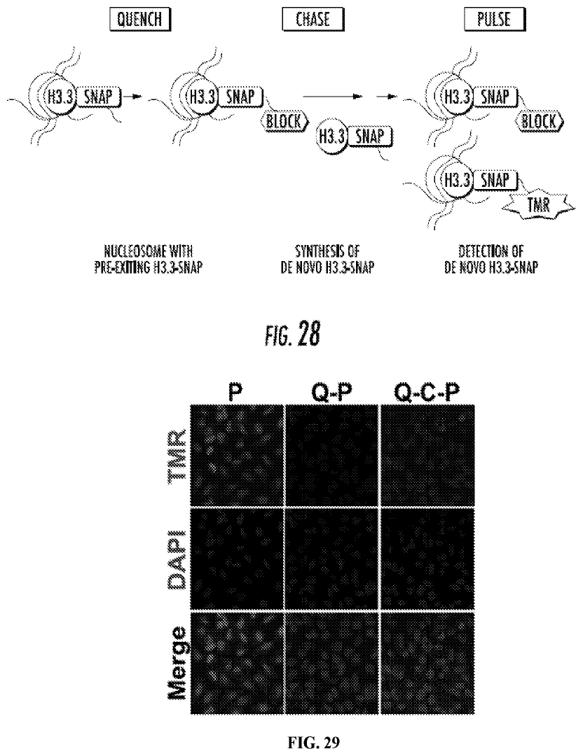

To identify the role of O-GlcNAcylation of histone variant h3.3 chaperone protein, HIRA, on transdifferentiation. The effects of O-GlcNAcylation on proteins that may be involved in the epigenetic regulation of transdifferentiation can be assessed. Nucleosome turnover is known to be tightly interrelated with DNA accessibility, and histone variant h3.3 is a hallmark of regulatory regions in the mammalian genome and is required to establish open chromatins. HIRA is the chaperone for h3.3, it forms a complex with other subunits, including ASF1. UBN, and CABIN, and is responsible for h3.3 deposition on the chromatin. OGT interacts with and O-GlcNAcylates HIRA, both of which are critical for the formation of the HIRA-h3.3 complex and h3.3 deposition and its dependent gene activation. To determine if the alteration in HIRA O-GlcNAcylation will alter h3.3 deposition during transdifferentiation. OGT and OGA knockdown cells will be used to determine the role of O-GlcNAcylation in the interaction between HIRA complex subunits and h3.3 during transdifferentiation by performing immunoprecipitation studies. The role of HIRA in transdifferentiation with fibroblasts that have HIRA knocked down or overexpressed can be determined. The h3.3-SNAP tagging system and the quench-pause-chase method to determine if h3.3 deposition is enhanced during transdifferentiation and regulated by OGT or OGA knockdowns can then be use. The SNAP tag is a genetically modified version of the human 06-alkylguanine DNA-alkyltransferase (hAGT). The SNAP-tag-fused protein then can be labeled with the cell-permeable SNAP-tag substrates incorporating various labels. This established approach is used to study histone protein turnover, recycling, and de novo deposition combined with quench-chase-pulse experiments (FIG. 28). The SNAP-h3.3 fusion protein will be overexpressed in the OGT or OGA KD or control fibroblasts, then a non-fluorescent snap blocker, which quenches pre-existing SNAP-h3.3, will be delivered. The newly synthesized SNAP-h3.3 will be expressed following the chase phase (FIG. 29. Q-C-P). Thereafter, the de novo deposited SNAP-h3.3 will be pulse-labeled with the TMR-Star, which is a readily detectable and quantifiable red-fluorescent substrate. To determine the effect of O-GlcNAcylation on old h3.3 retention. TMR pulsing will perform followed by a few hours of chasing and the remaining TMR signal will reflect the H3.3 retention. This method was established in Hela cells (FIG. 29) and will apply similar strategies in BJ fibroblasts that undergo transdifferentiation.

It is expected that the OGT KD will impair, whereas OGA KD will enhance HIRA-h3.3 complex formation during transdifferentiation. Furthermore. HIRA KD will impair transdifferentiation. It is expected that h3.3 deposition, either through h3.3 de novo deposition or retention (which processes may be mediated by different mechanisms), to be enhanced during transdifferentiation. Another subunit of the HIRA complex, UBN, is also known to be O-GlcNAcylated. If no changes with the HIRA knockdown, then the focus will be on the role of O-GlcNAcylation on UBN. Although HIRA is a top candidate as the O-GlcNAcylation substrate during transdifferentiation.

Determine the significance of O-GlcNAcylation in revascularization during limb ischemia recovery. Data shows that there is an increase in O-GlcNAcylation during recovery from limb ischemia (FIGS. 6A-6B), and modulation O-GlcNAcylation by inhibitors decreases ischemia recovery (FIGS. 7A-7B), suggesting O-GlcNAcylation may play a regulatory role in revascularization. Therefore, these experiments can build upon this to determine the effects of O-GlcNAcylation on ischemia-reperfusion involving regulation of transdifferentiation. O-GlcNAcylation can enhance in vivo transdifferentiation and therefore revascularization. Thus, fibroblast lineage tracing strategies, the limb ischemia model, and conditional knockout of OGT/OGA approaches in mice can be used to test this.

### Experimental:

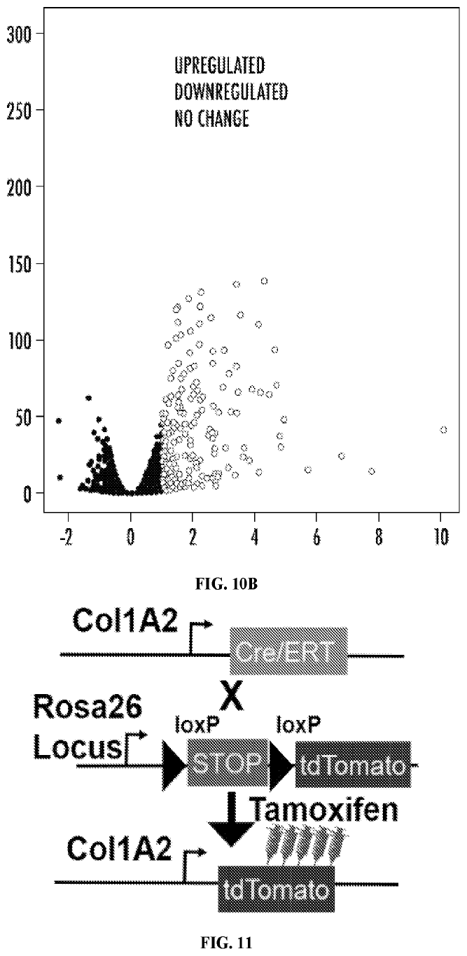

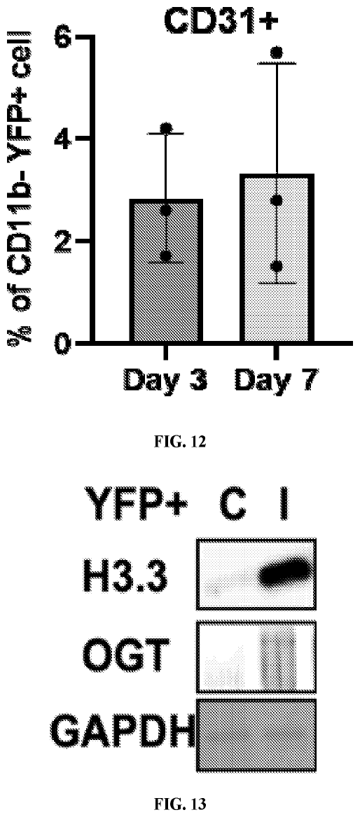

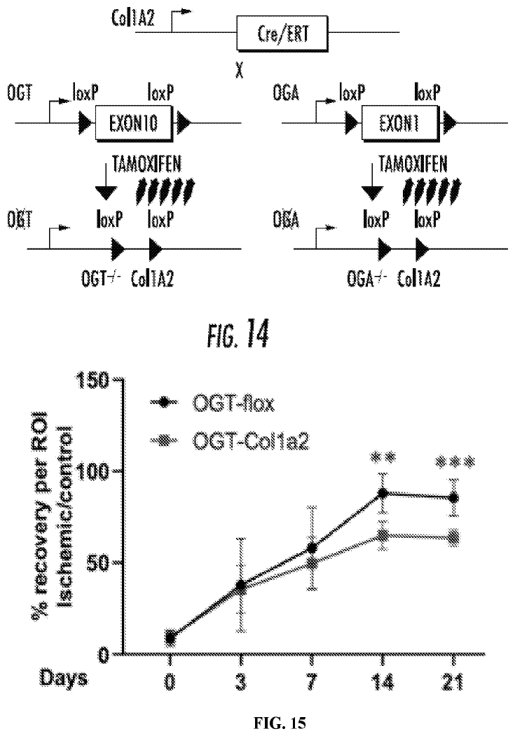

Identify the dynamics of O-GlcNAcylation during transdifferentiation in the ischemic hindlimb model. To firstly identify if O-GlcNAcylation level also increases during transdifferentiation in vivo, its level can be assessed in the fibroblast-derived cells from the lineage tracing mice. Col1A2-Cre/ERT: R26R-tdTomato (Swonger, J. M., et al., Differentiation, 2016. 92 (3): p. 66-83; Currie, J. D., et al., Biol Open, 2019. 8 (7); and Li. I. M. H., et al., Methods Mol Biol, 2017. 1627: p. 139-161), in which the tdTomato labeling on Col1A2 expressing fibroblast is induced by tamoxifen (FIG. 11). Labeling efficiency can be optimized before introducing limb ischemia. Limb muscle tissue can be collected at different time points (1, 2, or 3 weeks after 1 mg/mice/day tamoxifen IP injection for 5 consecutive days). Single cells can be isolated through tissue dissociation through liberase/collagenase digestion which will then be analyzed using flow cytometry for tdTomato red, and condition that gives the highest tdTomato signal can be chosen to perform the subsequent experiments. Limb ischemia can be introduced into the mice and single cells isolated from muscle tissues of operated and non-operated limbs can be isolated on days 3, 7, 14, and 21 days post-surgery. CD31+tdTomato+ cells, which are considered as iECs, can be quantified using flow cytometry. OGT, OGA, O-GlcNAc, HIRA, H3.3 can be quantified in the sorted tdTomato+fibroblast progeny cells. Data with another fibroblasts lineage-tracing mice, FSP1-Cre: R26R-EYFP, where originally discovered the transdifferentiation phenomenon in the ischemic limb recovery (Meng, S., et al., Circulation, 2020. 142 (17): p. 1647-1662), shows the absence of CD11b−YFP+CD31+ cells (IEC) in the limbs before surgery, but this population increased rapidly post-surgery on days 3 and day 7 (FIG. 12) (CD11b is used to negatively select the FSP1 expressing myeloid-monocytic lineage cells). CD11b−YFP+ cells isolated from ischemic limb (I) at 3 days post-surgery showed a significant increase in H3.3 and OGT compared with non-operated limbs (C) suggesting there was an activation of the OGT-H3.3 pathway in the fibroblast progenies during transdifferentiation (FIG. 13). Similar experiments and assessments can be performed in the Col1A-tdTomato strain as in the FSP-EYFP strain. The identified epigenetic-transcriptional networks will also be screened and confirmed in this model.

Level in the fibroblast-derived cells from the lineage tracing mice can be assessed. The mouse model used in the preliminary studies is a constitutive FSP1-YFP model, which may not capture the rapid changes during vascular regeneration, a tamoxifen-inducible Col1A2-Cre/ERT-tdTomato fibroblast lineage tracing model can be used to confirm studies by pulse-chase labeling. Femoral artery ligation surgery will be performed on the mice to induce ischemia in the hindlimbs. On days 0, 3, 7, 14, and 21 post-surgeries, the mouse hindlimb muscle tissue will be isolated and disaggregated cells will be analyzed by flow cytometry. To confirm the transdifferentiation phenomenon in vivo in this tamoxifen-inducible lineage tracing model, the levels of endothelial markers (CD31, CD144, etc.) and fibroblast markers (PDGFRA, CD90), etc.) in the tdTomato+ and tdTomato-cells at different time points post-surgery by flow cytometry and immunofluorescent staining will be evaluated. To characterize metabolic and epigenetic changes, including the O-GlcNAcylation dynamics and HIRA-h3.3 pathway activities, during transdifferentiation over time, tdTomato+ and tdTomato-cells will be isolated and the total levels of OGT. OGA, O-GlcNAcylation, and the level of HIRA that has been O-GlcNAcylated will be determined. IP experiments to characterize the interactions among OGT, the subunits of the HIRA complex, and h3.3 in those different cell populations can also be perform.

It is expected to observe a similar transdifferentiation phenomenon in the tamoxifen-inducible Col1A2-Cre/ERT-tdTomato lineage tracing mice as observed in FSP-YFP mice. Alternatively, the ability of the cell fate transition may be predetermined in the more primitive stage of life in the mice, with only a small subset of cells (expressing FSP1 but not Col1A2) within the heterogeneous fibroblast population possessing regenerative capacity. additional lineage tracing mice, PDGFRA-Cre/ERT-tdTomato mice, will be then include or create inducible FSP1-Cre mice to further address those possibilities. It is also expected that in the tdTomato+ cells in the ischemic limb. O-GlcNAcylation will increase rapidly but decrease to normal levels by recovery. HIRA O-GlcNAcylation is expected to increase, and the interaction between OGT. HIRA, and h3.3 will be enhanced. It is expected to detect higher levels of the genes that are identified in the ATAC seq in the tdTomato+ cells from the ischemic limb. In the case that HIRA O-GlcNAcylation doesn't change and the HIRA-h3.3 pathway is not activated during transdifferentiation, then focus on other candidates identified with the quantitative and site-specific O-GlcNAcomics.

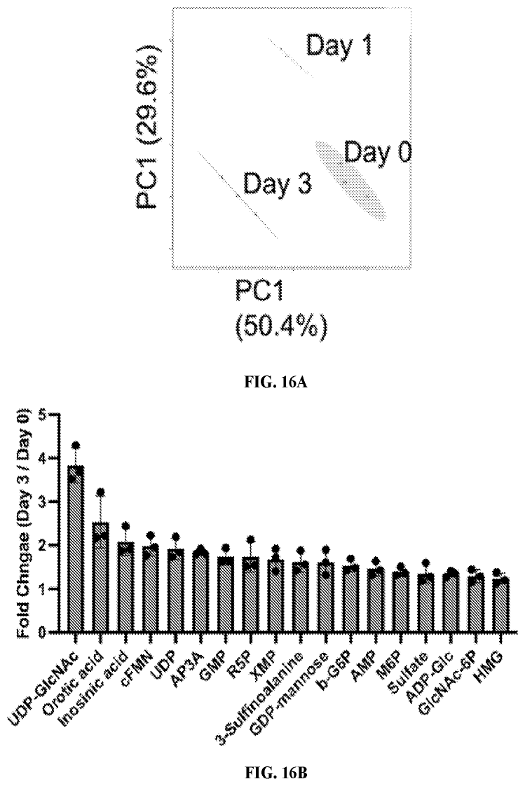

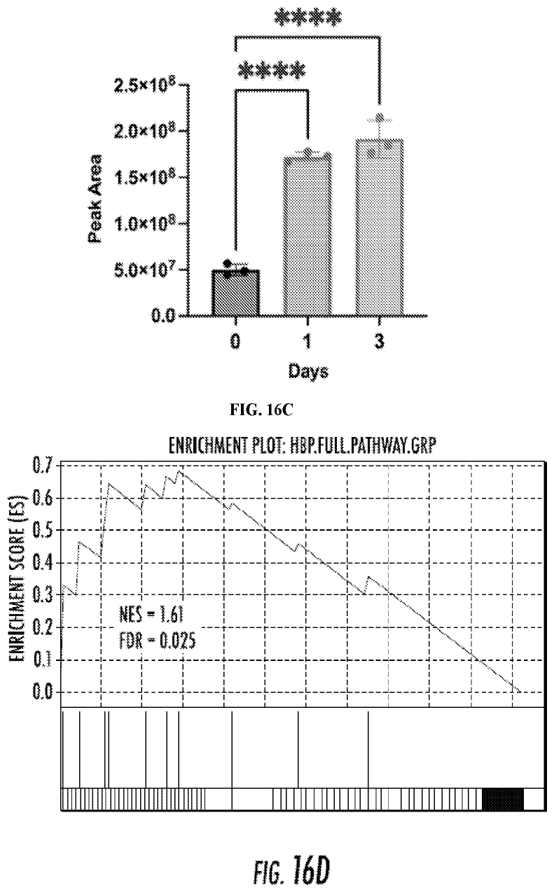

Test the effect of OGT/OGA knockout in transdifferentiation in vivo, Generate the fibroblasts specific OGT or OGA knockout mice by crossing the OGT flox-flox mice flanking exon 10) (Jackson labs) or OGA flox-flox mice flanking exon 1 (from collaborator Dr. Hanover from NIDDK) with tamoxifen-inducible Col1a2-Cre/ERT mice (FIG. 14). For the Col1A2 Cre-driven knockout mice, first optimize the knockdown induction by testing the OGT/OGA level in the isolated mice fibroblasts at different days after 1 mg/mice/day tamoxifen injection for 5 consecutive days. The optimal time points that obtained the best knockdown efficiency can be used for experiments to look at transdifferentiation in the ischemic hindlimb model. Comparisons of revascularization can be made between the control group (OGT or OGA flox-flox mice injected with tamoxifen) and the knockout group (OGT or OGA-Col1a2 mice injected with tamoxifen) using laser doppler imaging with monitoring at days 3, 7, 14, 21 post-surgery. Vascular density measurements can also be made in mice 21 days post-surgery through immunohistochemistry on tissue sections to detect CD31+ endothelial cells. Limb tissues can be analyzed at days 0, 3, 7, 14, and 21 post-surgery for OGT-HIRA-H3.3 pathway activities or other candidate pathways identified by O-GlcNAcomics and multi-omics, and comparisons can be made between the control and knockout mice. The gene encoding OGT resides on the X chromosome which is subject to complex mechanisms of dosage compensation in females. So only male mice of the appropriate genotypes can be used. However, both genders can be studied in OGA knockout mice. Studies using OGT-flox and OGT−/−Col1a2 male mice where limb ischemia was introduced 7 days after the last injection of tamoxifen showed a significant decrease in blood flow starting from day 14 post-surgery (n=6, P<0.01, **; P<0.001, ***) suggesting O-GlcNAcylation enhances revascularization partially through transdifferentiation (FIG. 15). Additional OGT−/−Col1a2 mice can be assessed for the ischemic recovery and OGT-HIRA-H3.3 pathway, and similar experiments and assessments can be performed in OGA−/−Col1A2 mice.

Results and alternative strategies: The lab previously identified the in vivo transdifferentiation phenomenon in the FSP1-Cre: R26R-EYFP lineage-tracing mice in the ischemic hindlimb mouse model (Meng, S., et al., Circulation, 2020. 142 (17): p. 1647-1662). While FSP1 has been suggested to be specific to fibroblasts it cannot be rule out the potential expression of this promoter in embryonic cells. While ensuring that endothelial cells lack the reporter expression, there is the potential that reprogramming may activate this promoter in more primitive stem cells, rather than transdifferentiation of fibroblasts. Therefore, the transdifferentiation process in tamoxifen regulated, Col1A2-tdTomato strain can also be analyzed, which can better capture the short-term cell fate transition and epigenetic changes. Based on data with FSP-EYFP mice, similar results can be expected in mice where there can be an acute activation in O-GlcNAcylation and OGT-HIRA-H3.3 signaling and iEC number, which can diminish when the revascularization is complete. While the data suggest that the increase can be captured in this model, if only one time point in which these proteins are elevated is observe, the time frame can be altered to include either daily or hourly time points to capture these changes. Also, there is the chance that transdifferentiation cannot be detected or the level is very low which can be due to the less abundant of Col1A2 expression in the limb fibroblasts or the O-GlcNAcylation modulation is not targeting this population, then the inducible FSP1-EYFP mice can be generated for this purpose. Alternatively, the single-cell multi-omics and analyze the signatures of cells within ischemic muscle tissue can be used, by which the population that has the most elevated O-GlcNAcylation can be determine. Based on the data, impairment or enhancement in revascularization after OGT or OGA is knocked out in fibroblasts can be expect, and the OGT-HIRA-H3.3 pathway can be impaired in the ischemic tissue if OGT is knocked out while the opposite can be observed in OGA knockout mice. Based on the data it is expected to see impairment or enhancement in revascularization after OGT or OGA is knocked out in fibroblasts. The OGT-HIRA-h3.3 pathway can be impaired in the ischemic tissue if OGT is knocked out while the opposite can be observed in OGA knockout mice. However, Col1A2 may mark different fibroblast cells than the Fsp1-expression fibroblasts, which may potentially result in no significant difference between OGT/OGA-Col1a2-Cre/ERT and control mice. If Col1A2 Cre is proven to be less efficient in fibroblasts labeling in limb, inducible OGT−/−FSP1 and OGA−/−FSP1 mice can be generated. Additional tamoxifen-inducible OGT or OGA knockout can be used by crossing OGT/OGA-flox/flox mice with PDGFRA-Cre/ERT mice or create the OGT/OGA knockout mice with inducible Fsp1 Cre. O-GlcNAcylation enhancement of revascularization may function partially through mechanisms other than transdifferentiation, such as simply inducing angiogenesis. OGT−/−CDH5 or OGA−/−CDH5 mice can be generated to assess their effects on recovery from limb ischemia. O-GlcNAcomic and single-cell multi-omic analysis of the limb tissues at different points of healing can allow to identify the tentative molecular mechanism for this transdifferentiation process. scRNAseq can also be used to identify changes in OGT/OGA knockout in fibroblasts in the iEC clustering in ischemic limb tissue during recovery which is echoing previous finding (Meng, S., et al., Circulation, 2020). 142 (17): p. 1647-1662). Moreover, O-GlcNAcylation enhancement of revascularization may function partially through mechanisms other than transdifferentiation, such as simply inducing angiogenesis. OGT−/−CDH5 or OGA−/−CDH5 mice can be generated to assess their effects on recovery from limb ischemia.

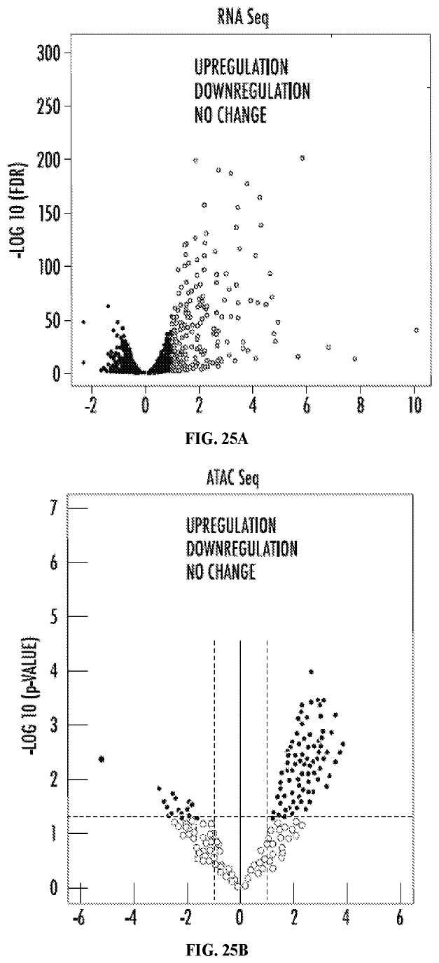

To characterize the activity of the OGT-HIRA-h3.3 pathway in vascular recovery in humans, limb tissue from patients undergoing amputation due to acute limb ischemia, e.g., embolic events, tumors, or trauma can be collected. patients with pre-existing vascular disease or diabetes can be excluded, as it is of interest to assess the role of the pathway in the normal human response to ischemia. The fibroblast from the proximal or distal tissue can be isolated and compare the O-GlcNAcylation levels and the integrity of the HIRA-h3.3 complex. In studies, control and ischemic tissue samples from amputated limbs from a patient without pre-existing vascular disease, who incurred acute limb ischemia due to a cardiogenic embolism (non-PAD patient) and from a patient with pre-existing chronic vascular disease, i.e., critical limb ischemia (CLI) were collected. At the time of amputation, tissue was collected in the region of ischemia, and a region of non-ischemic tissue, immediately below the site of amputation. Analysis of these de-identified tissues showed that O-GlcNAcylation levels are significantly upregulated in the ischemic tissue compared with non-ischemic control in the non-PAD patient (FIG. 27), which matches the findings in the mice recovering from hindlimb ischemia (FIGS. 6B, 19B, 19D, and 26). Intriguingly, in the CLI patient, a diminished O-GlcNAcylation in the ischemic site was observed (FIG. 11 right). It can also be confirmed if O-GlcNAcylation is dysregulated in CLI and if this dysregulation contributes to the impaired angiogenesis that is observed in CLI.

Based on the data (FIG. 27), it is expected to confirm that O-GlcNAcylation is increased in ischemic versus perfused human tissue. An enhanced O-GlcNAcylation and HIRA-h3.3 complex integrity in the patient fibroblasts isolated from the ischemic area compared with the control is expected. Isolated cells can also be cultured to assess the role of O-GlcNAcylation in transdifferentiation and angiogenesis in vitro, as previously described above in the mouse studies. It would be of interest to address the possibility of O-GlcNAcylation regulation in other cell types, e.g., endothelial cells.

### Example 2

The ability to restore or enhance the microvasculature would be a major advancement in regenerative medicine and cardiovascular therapies. Angiogenic transdifferentiation from fibroblasts to endothelial cells has been suggested to directly contribute to microvasculature repair and limb perfusion restoration after ischemia. The previous studies uncovered that regulating cell metabolism to facilitate transdifferentiation may be a novel strategy for treating ischemia and vascular regeneration. To comprehensively characterize the metabolic component that contributes to transdifferentiation, with the global metabolic profiling, uridine diphosphate N-acetylglucosamine (UDP-GlcNAc), and O-GlcNAcylation may be also involved in transdifferentiation. O-GlcNAcylation is an essential post-translational protein modification, which uses UDP-GlcNAc as the substrate to directly alter protein activity. O-GlcNActransferase (OGT) and O-GlcNAcase (OGA) are the pair of enzymes that add or remove this protein modification respectively. By using in vitro transdifferentiation experiment, the pharmacological or genetic inhibition of OGT in fibroblasts impairs transdifferentiation while OGA inhibition enhances transdifferentiation. A fibroblasts lineage tracing study showed that O-GlcNAcylation inhibition decreased the transdifferentiation population in the hindlimb ischemia model. By using the fibroblast conditional knockout strategy, OGT KO mice showed impaired vascular recovery in the hindlimb ischemic model while OGA KO mice showed enhanced revascularization supporting that O-GlcNAcylation is critical for transdifferentiation and vascular regeneration. Mechanistically, O-GlcNAcylation on H3.3 chaperon protein HIRA is essential for transdifferentiation and de novo H3.3 deposition onto the chromatin of active genes. O-GlcNAcylation enhances transdifferentiation and vascular regeneration by regulating the H3.3 deposition to facilitate cell fate transition.

### INTRODUCTION

The prevalence of ischemic vascular disorders, such as myocardial infarction, cerebrovascular disease, peripheral vascular disease, and microcirculatory disorder is increasing worldwide. Peripheral artery disease (PAD) occurs in about 12% of the U.S. adult population, and in the most severe form of critical limb ischemia (CLI), is associated with a mortality rate of 20% to 26% within 1 year of diagnosis. Endovascular procedures and surgical bypass often fail and lead to amputation in the absence of efficacious medical treatment. Ischemia-induced neovascularization is critical for perfusion recovery; however, angiogenic therapies have largely failed in treating the complications (e.g. ischemic ulcers) of CLI. New strategies are desperately needed to restore the vasculature in those patients.

Transdifferentiation, also called direct cell reprogramming, is the process that one somatic cell type is reprogrammed directly into another somatic cell type without transition through an induced pluripotent state. It has emerged as an attractive approach for tissue repair where there are shortages of certain types of cells that can be replenished by transdifferentiation from cells that are abundant in the body. While the most popular method for transdifferentiation is overexpressing transcriptional factors that are known to be important for the desired cell lineage via lentiviral vectors, genome integration is a safety concern limiting its application in human subjects. A stem-stage-free, transgene-free, pharmacological agents-only method for angiogenic transdifferentiation from fibroblast to endothelial cells in vitro was previously developed. In this two-stage protocol, first activate innate immune signaling with the TLR3 agonist polyinosinic: polycytidylic acid (Poly I:C), which causes global changes in the balance of epigenetic modifiers and epigenetic plasticity, a process termed “transflammation”. Then the cells will further undergo transdifferentiation under the guidance of endothelial lineage growth factors in the medium. The induced endothelial cells generated from this protocol are functionally and transcriptionally comparable with genuine endothelial cells. More recently, the group discovered evidence of transdifferentiation in situ with fibroblast lineage tracing mouse model in a murine limb ischemia model. The in vitro and in vivo data suggests that transdifferentiation may be a target to potentiate vascular recovery and tissue regeneration.

The regulation of cell-specific transcriptional networks is accomplished by an epigenetic program via chromatin-modifying enzymes, whose activity is directly dependent on intermediary metabolites. Changes in acetyl-CoA, SAM, ATP, NAD+, FAD, a-KG, and many other metabolites couple chromatin-dependent gene regulation with the metabolic state of the cell to regulate cell plasticity and ultimately control physiological and pathological processes. However, little is known about whether metabolism plays a role in transdifferentiation. Previous work identified a glycolytic switch is required for transdifferentiation. This glycolytic shift is associated with citrate export from the mitochondria to the nucleus, which provides the substrate for acetyl-CoA synthesis in the nucleus, and its use in histone acetylation to increase DNA accessibility. However, the comprehensive profile of the metabolic changes during this process, and whether other metabolites contribute to epigenetic plasticity in transdifferentiation-driven revascularization is not clear. In this study, global metabolomic screen on cells undergoing transdifferentiation was performed and identified the UDP-GlcNAc, the substrate of post-translational modification O-GlcNAcylation, as the most up-regulated metabolites.

O-GlcNAcylation is an essential post-translational protein modification, which often directly alters their activity. O-GlcNAc-transferase (OGT) and O-GlcNAcase (OGA) are the pair of enzymes that add or remove this protein modification respectively. Accumulating data have revealed that O-GlcNAcylation is essential in the modulation of chromatin remodeling by modifying histone tails and epigenetic modifiers. Studies showed that O-GlcNAcylation confers protection following acute ischemic reperfusion and other types of cardiovascular injuries by improving post-ischemic contractile function recovery in the heart. However, its role in limb ischemia recovery and transdifferentiation in ischemic syndromes requires further investigation. In this study, it was determined the role of O-GlcNAcylation in promoting transdifferentiation and revascularization using in vitro and in vivo models.

### Results

### UDP-GlcNAc Level is Significantly Upregulated During Transdifferentiation.

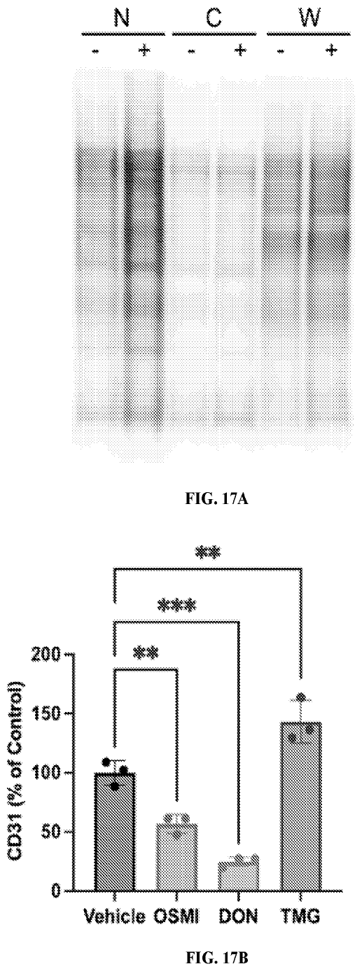

To profile the global metabolic changes during transdifferentiation, untargeted metabolomics with BJ human fibroblast cells undergoing transdifferentiation protocol was performed and treated with 30 ng/ml Poly I:C with the induction medium for 0, 1, and 3 days. Over 100 metabolites were identified from the LC-MS-based metabolomic analysis. Principal Component Analysis shows clear separation in different time point groups and consistency among the triplicates of each time point (FIG. 16A). Among the 18 significant up-regulated metabolites (std<0.05) on day 3 in comparison with day 0. UDP-GlcNAc is the top one that has the greatest fold change (FIG. 16B), and it consistently increases on day 1 and day 3 (FIG. 16C). UDP-GlcNAc is the substrate for O-GlcNAcylation and is the end-product of the hexosamine biosynthesis pathway (HBP). A pathway analysis among the significantly changed metabolites was performed and found that the HBP pathway metabolites are significantly enriched which confirmed the importance of HBP and O-GlcNAcylation in the transdifferentiation (FIG. 16D).

### O-GlcNAcylation is Elevated and Required During Transdifferentiation In Vitro

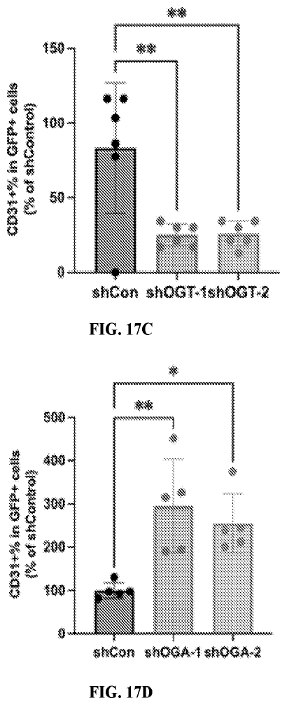

To determine if the increase in UDP-GlcNAc is associated with an increase in O-GlcNAcylation, cells were treated with Poly I:C or vehicle for 3 days, then the O-GlcNAcylated proteins in the nuclear (N), cytoplasmic (C), or whole-cell (W) lysates were enzymatically labeled with azido-modified galactose (GalNAz), which were further detected by click-it chemistry using a biotinylated alkyne and Western. The results comparing the Poly I:C-treated and untreated groups show that there is an elevation in O-GlcNAcylation during transdifferentiation which primarily occurs in nuclear proteins (FIG. 17A) suggesting the potential role of O-GlcNAcylation in epigenetic regulation.

To define if the elevation of O-GlcNAcylation is required for transdifferentiation, inhibitors of O-GlcNAcylation including OSMI (an inhibitor for OGT) and DON (an inhibitor for GFAT that acts upstream in the hexosamine biosynthesis pathway and reduces O-GlcNAcylation) or an activator of O-GlcNAcylation. Thiamet G (TMG) (an OGA inhibitor that enhances O-GlcNAcylation), were delivered to BJ fibroblasts undergoing transdifferentiation. The number of CD31 expressing iECs generated during transdifferentiation decreases when exposed to OSMI or DON and increases when fibroblasts are exposed to TMG (FIG. 17B). Then OGT or OGA were knocked down using shRNA in the BJ fibroblasts (FIG. 17C) and performed transdifferentiation protocol in vitro. The data revealed that transdifferentiation was impaired by blocking OGT and was enhanced by blocking OGA (FIG. 17D). These data suggest that transdifferentiation is regulated by O-GlcNAcylation.

O-GlcNAcylation is Increased During Recovery from Ischemia

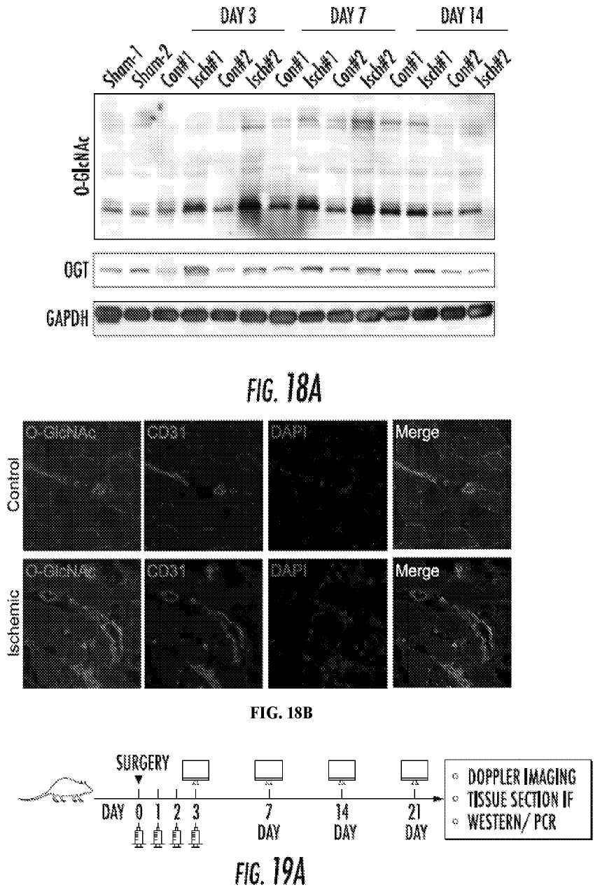

Previous work suggested that angiogenic transdifferentiation directly contributes to vascular regeneration in a hindlimb ischemia mouse model. Then this model was used to determine the role of O-GlcNAcylation in transdifferentiation and revascularization in vivo. First, with WT C57BL6 mice, the level of O-GlcNAcylation at different time intervals over the 14-day post-surgery recovery in the gastrocnemius tissue was profiled. Intriguingly. Western blotting (FIG. 18A) of hindlimb tissues revealed a substantial increase in O-GlcNAcylation and OGT expression in the ischemic limb by comparison to the control (unoperated limb) at day 3 and day 7 but had resolved by day 14, at which time perfusion had substantially recovered (FIG. 2D). Immunofluorescent staining also showed a significant increase in O-GlcNAcylation in the ischemic limb in comparison with the control limb 3 days post-surgery (FIG. 18B). Altogether, this evidence suggests that O-GlcNAcylation is increased at the acute phase and dynamically regulated with hindlimb ischemia.

### O-GlcNAcylation is Required for the Vascular Recovery

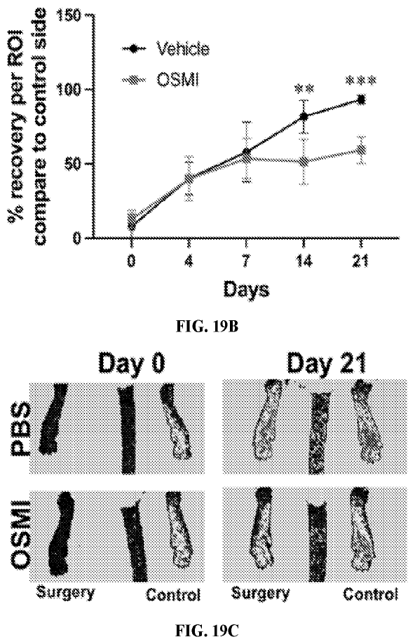

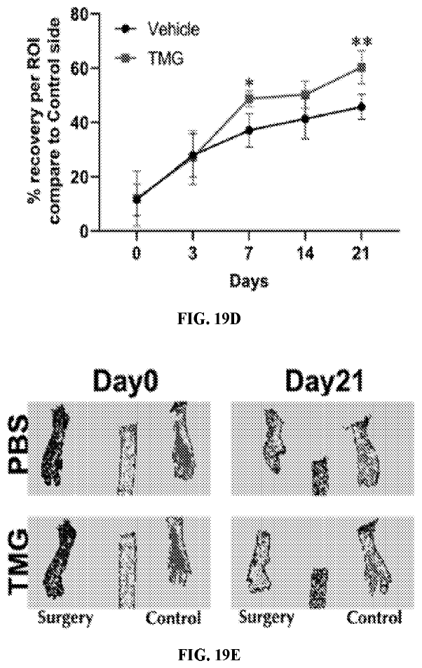

To determine if O-GlcNAcylation manipulations can modulate the overall effect of vascular recovery from limb ischemia. WT C57BL/6 mice were treated with the O-GlcNAcylation inhibitor. OSMI4 (10 mg/kg), or O-GlcNAcylation enhancer. TMG (10 mg/kg) 0, 1, 2 days post-surgery (FIG. 19A). The Doppler imager was used to monitor the data showed that OSMI impaired recovery (FIGS. 19B&19C), while TMG enhanced recovery of blood flow post femoral artery ligation (FIGS. 19D&19E). Vascular density measurement by CD31 immunofluorescent staining data suggests that O-GlcNAcylation enhances overall vascular regeneration during the recovery from limb ischemia.

### O-GlcNAcylation Enhances Transdifferentiation In Vivo

To determine if O-GlcNAcylation enhances revascularization through transdifferentiation, fibroblasts lineage tracing Fsp1-Cre: R26R-EYFP mice strain were utilized where it was previously observed the in situ transdifferentiation phenomenon (FIG. 20A). In this model, all the fibroblasts expressing Fsp1 (fibroblast-specific protein1) are marked with YFP. With this model, a significant increase in the FSP-expressing cell progeny in the ischemic limb was identified compared with the sham-operated limb 3 days after the femoral artery ligation was perform (FIG. 20A) and compare the YFP+ cells over the different time points (FIG. 20A). The percentage of the YFP+CD31+CD11b-cell population which is considered the transdifferentiation population was analyzed further. The data showed that the YFP+CD31+CD11b-population expanded rapidly on day 3 and day 7 post-surgery (FIG. 20B) which was confirmed by Immunofluorescent staining (FIG. 20C). To characterize the metabolic status of the fibroblast progeny cells, the YFP+ and the YFP-non-fibroblast progeny cells with CD11 negative selection were sorted out to exclude the FSP-expressing macrophages in both the control and ischemic limbs. Western results showed a dramatic accumulation of O-GlcNAcylation in the YFP+ cells, especially in the ischemic limb (FIG. 20D). OGT level changes also reflect the changes in O-GlcNAcylation (FIG. 20D). To determine the role of the O-GlcNAcylation on the transdifferentiation, the Fsp1-Cre: R26R-EYFP mice that are undergoing vascular recovery with OSMI were treated, and the transdifferentiation population in the limb muscle tissue 7 days post-surgery was analyzed. The data showed that the OSMI treatment significantly reduced the transdifferentiation population (FIG. 20D) suggesting that O-GlcNAcylation could enhance revascularization through transdifferentiation.

Fibroblast-specific O-GlcNAcylation manipulation regulates vascular recovery

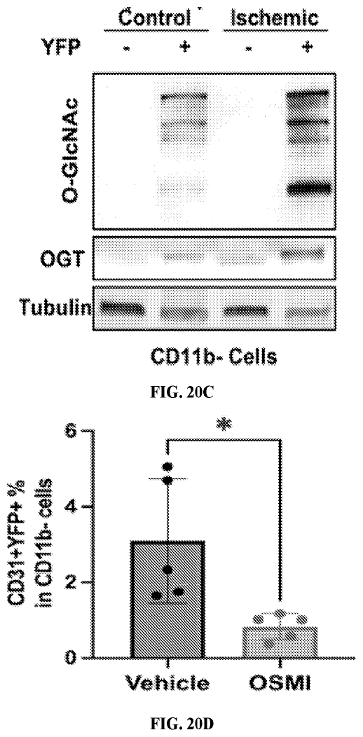

To further elucidate the role of O-GlcNAcylation in transdifferentiation and revascularization in vivo, fibroblast-specific OGT or OGA knockout mice were generated by crossing tamoxifen-inducible collagen Type I Alpha 2 Chain (Col1A2) with OGT or OGA flox mice (FIG. 14). Mice were injected with 1 mg 4-OHT for 5 consecutive days to induce Col1a2-cre specific knockout of OGT or OGA. The OGT or OGA flox mice injected with 4-OHT were used as the control mice. The knockdown efficiency was checked in the tail tip fibroblasts from the knockout mice and control mice, the hindlimb ischemia mode 7 days after the last 4-OHT injection on the mice was performed, and the vascular recovery using Doppler imaginer was monitored. The data showed that the fibroblast-specific OGT knockout impairs while OGA knockout enhances vascular recovery (FIG. 21A-21C). CD31 immunofluorescent staining on the limb tissue sections also confirmed that OGT knockout mice have reduced while OGA knockout mice have enhanced vascular density (FIG. 21D-21F).

### HIRA-H3.3 Signaling is Activated During Transdifferentiation and Vascular Recovery

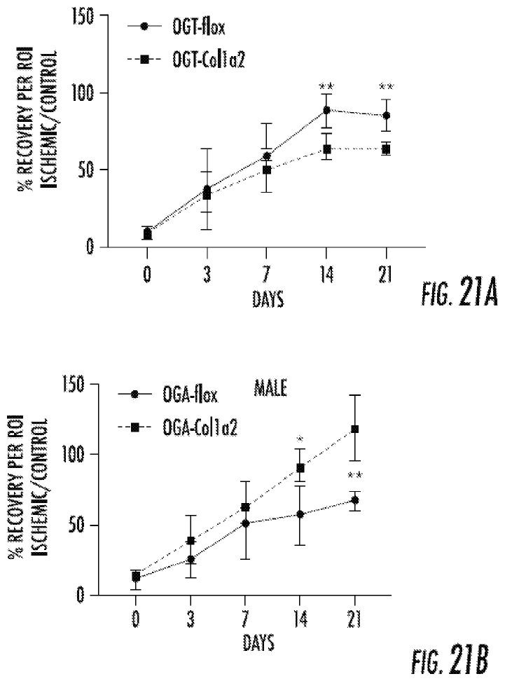

The histone variant h3.3 is a replacement for its canonical form of h3.1 and h3.2 in the nucleosome. Its deposition is usually associated with transcription activation and enhanced DNA accessibility. The complex that is responsible for h3.3 deposition is called HIRA complex which consists of HIRA. UBN, and cabin as the histone chaperone proteins. The HIRA O-GlcNAcylation is known to be critical for its function in H3.3 deposition. During the vascular recovery in the WT mice, the level of HIRA complex subunits, including ASFIA. HIRA. UBN, as well as the H3.3 deposition are increased (FIG. 22A). The HIRA-H3.3 pathway in the fibroblast progeny cells is activated, especially in the limbs recovering from ischemia 3- and 7-days post-surgery compared with the non-YFP+ cells (FIG. 22B). Interestingly, the differences between the YFP+ cells and the YFP-cells in the HIRA-H3.3 signaling are reduced on day 14, consistent with the diminished difference in O-GlcNAcylation level, when the revascularization is almost complete, suggesting a very dynamic regulation of H3.3. An in vitro system to test whether the HIRA-H3.3 pathway is activated during transdifferentiation was used. The immunoprecipitation using HIRA antibody showed that the HIRA interaction with UBN and H3.3 are both enhanced after Poly I:C treatment suggesting a potentially important role of the HIRA-H3.3 pathway in transdifferentiation.

### H3.3 Deposition is Enhanced During Transflammation.

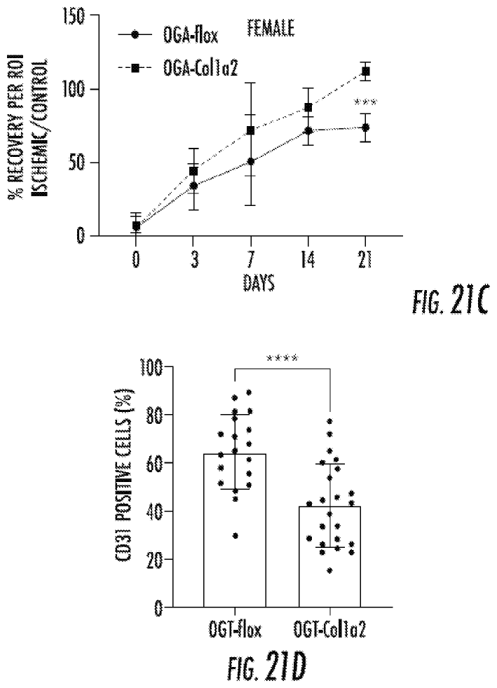

To test whether innate immune activation enhances the H3.3 deposition in the chromatin, the H3.3-SNAP tagging system was generated where the newly synthesized H3.3 will be detected by fluorescent TMR-Star labeling with quench-chase-pulse strategy. The results showed that the Poly I:C enhances the H3.3 deposition which is impaired by OSMI (FIG. 23A-23C). The data suggested that the O-GlcNAcylation enhances the transdifferentiation by promoting H3.3 deposition.

### O-GlcNAcylation of HIRA Enhances Transdifferentiation.

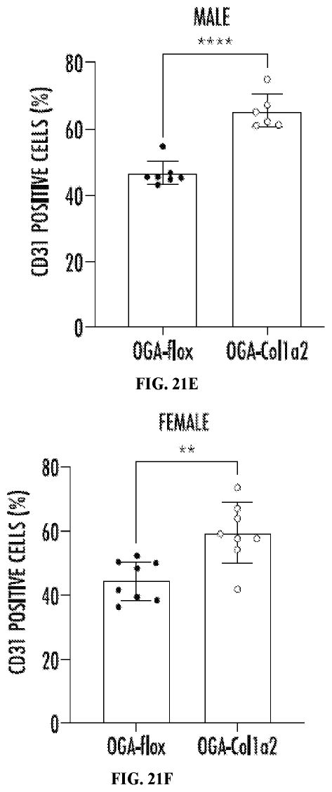

Previous studies have reported the O-GlcNAcylation sites on HIRA and suggested that the Serine 231 site is the most important O-GlcNAcylation site for the H3.3 deposition. Thus, first HIRA was knocked down in BJ fibroblasts using siRNA and found the global HIRA knockdown impairs transdifferentiation in vitro. Then, the S231 was overexpressed to A site mutated HIRA in the BJ fibroblast cells (FIG. 24) and the result showed that the S231A HIRA mutation overexpressed fibroblasts have impaired transdifferentiation compared with WT HIRA control suggested the O-GlcNAcylation at S231 HIRA is critical for transdifferentiation.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

### SEQUENCES

SEQ ID NO: 1 (human O-linked N-acetylglucosamine (GlcNAc) transferase-
Gene ID 8473)
ATGGCGTCTTCCGTGGGCAACGTGGCCGACAGCACAGAACCAACGAAACGTATG
CTTTCCTTCCAAGGGTTAGCTGAGTTGGCACATCGAGAATATCAGGCAGGAGATT
TTGAGGCAGCTGAGAGACACTGCATGCAGCTCTGGAGACAAGAGCCAGACAAT
ACTGGTGTGCTTTTATTACTTTCATCTATACACTTCCAGTGTCGAAGGCTGGACAG
ATCTGCTCACTTTAGCACTCTGGCAATTAAACAGAACCCCCTTCTGGCAGAAGCT
TATTCGAATTTGGGGAATGTGTACAAGGAAAGAGGGCAGTTGCAGGAGGCAATT
GAGCATTATCGACATGCATTGCGTCTCAAACCTGATTTCATCGATGGTTATATTAA
CCTGGCAGCCGCCTTGGTAGCAGCGGGTGACATGGAAGGGGCAGTACAAGCTTA
CGTCTCTGCTCTTCAGTACAATCCTGATTTGTACTGTGTTCGCAGTGACCTGGGG
AACCTGCTCAAAGCCCTGGGTCGCTTGGAAGAAGCCAAGGCATGTTATTTGAAA
GCAATTGAGACGCAACCGAACTTTGCAGTAGCTTGGAGTAATCTTGGCTGTGTTT
TCAATGCACAAGGGGAAATTTGGCTTGCAATTCATCACTTTGAAAAGGCTGTCAC
CCTTGACCCAAACTTTCTGGATGCTTATATCAATTTAGGAAATGTCTTGAAAGAG
GCACGCATTTTTGACAGAGCTGTGGCAGCTTATCTTCGTGCCCTAAGTTTGAGTC
CAAATCACGCAGTGGTGCACGGCAACCTGGCTTGTGTATACTATGAGCAAGGCC
TGATAGATCTGGCAATAGACACCTACAGGCGGGCTATCGAACTACAACCACATTT
CCCTGATGCTTACTGCAACCTAGCCAATGCTCTCAAAGAGAAGGGCAGTGTTGC
TGAAGCAGAAGATTGTTATAATACAGCTCTCCGTCTGTGTCCCACCCATGCAGAC
TCTCTGAATAACCTAGCCAATATCAAACGAGAACAGGGAAACATTGAAGAGGCA
GTTCGCTTGTATCGTAAAGCATTAGAAGTCTTCCCAGAGTTTGCTGCTGCCCATTC
AAATTTAGCAAGTGTACTGCAGCAGCAGGGAAAACTGCAGGAAGCTCTGATGCA
TTATAAGGAGGCTATTCGAATCAGTCCTACCTTTGCTGATGCCTACTCTAATATGG
GAAACACTCTAAAGGAGATGCAGGATGTTCAGGGAGCCTTGCAGTGTTATACGC
GTGCCATCCAAATTAATCCTGCATTTGCAGATGCACATAGCAATCTGGCTTCCATT
CATAAGGATTCAGGGAATATTCCAGAAGCCATAGCTTCTTACCGCACGGCTCTGA
AACTTAAGCCTGATTTTCCTGATGCTTATTGTAACTTGGCTCATTGCCTGCAGATT
GTCTGTGATTGGACAGACTATGATGAGCGAATGAAGAAGTTGGTCAGTATTGTGG
CTGACCAGTTAGAGAAGAATAGGTTGCCTTCTGTGCATCCTCATCATAGTATGCTA
TATCCTCTTTCTCATGGCTTCAGGAAGGCTATTGCTGAGAGGCACGGCAACCTGT
GCTTAGATAAGATTAATGTTCTTCATAAACCACCATATGAACATCCAAAAGACTTG
AAGCTCAGTGATGGTCGGCTGCGTGTAGGATATGTGAGTTCCGACTTTGGGAATC
ATCCTACTTCTCACCTTATGCAGTCTATTCCAGGCATGCACAATCCTGATAAATTT
GAGGTGTTCTGTTATGCCCTGAGCCCAGACGATGGCACAAACTTCCGAGTGAAG
GTGATGGCAGAAGCCAATCATTTCATTGATCTTTCTCAGATTCCATGCAATGGAA
AAGCAGCTGATCGCATCCATCAGGATGGAATTCATATCCTTGTAAATATGAATGGC
TATACTAAGGGCGCTCGAAATGAGCTTTTTGCTCTCAGGCCAGCTCCTATTCAGG
CAATGTGGCTGGGATACCCTGGGACGAGTGGTGCGCTTTTCATGGATTATATTATC
ACTGATCAGGAAACTTCGCCAGCTGAAGTTGCTGAGCAGTATTCCGAGAAATTG
GCTTATATGCCCCACACTTTTTTTATTGGTGATCATGCTAATATGTTCCCTCACCTG
AAGAAAAAAGCAGTCATCGATTTTAAGTCCAATGGGCACATTTATGACAATCGGA
TAGTTCTGAATGGCATCGACCTCAAAGCATTTCTTGATAGTCTACCAGATGTGAA
AATTGTCAAGATGAAGTGTCCTGATGGAGGAGACAATGCAGATAGCAGTAACAC
AGCTCTTAATATGCCTGTTATTCCTATGAATACTATTGCAGAAGCAGTTATTGAAAT
GATTAACCGAGGACAGATTCAAATAACAATTAATGGATTCAGTATTAGCAATGGA
CTGGCAACTACTCAGATCAACAATAAGGCTGCAACTGGAGAGGAGGTTCCCCGT
ACCATTATTGTAACCACCCGTTCTCAGTACGGGTTACCAGAAGATGCCATCGTATA
CTGTAACTTTAATCAGTTGTATAAAATTGACCCTTCTACTTTGCAGATGTGGGCAA
ACATTCTGAAGCGTGTTCCCAATAGTGTACTCTGGCTGTTGCGTTTTCCAGCAGT
AGGAGAACCTAATATTCAACAGTATGCACAAAACATGGGCCTGCCCCAGAACCG
TATCATTTTTTCACCTGTTGCTCCTAAAGAGGAACACGTCAGGAGAGGCCAGCTG
GCTGATGTCTGCTTGGACACTCCACTCTGTAATGGGCACACCACAGGGATGGATG
TCCTCTGGGCAGGGACCCCCATGGTGACTATGCCAGGAGAGACTCTTGCTTCTC
GAGTTGCAGCATCCCAGCTCACTTGCTTAGGTTGTCTTGAGCTTATTGCTAAAAA
CAGACAAGAATATGAAGACATAGCTGTGAAGCTGGGAACTGATCTAGAATACCT
GAAGAAAGTTCGTGGCAAAGTCTGGAAGCAAAGAATATCTAGCCCTCTGTTCAA
CACCAAACAATACACAATGGAACTAGAGCGGCTCTATCTACAGATGTGGGAGCA
TTATGCAGCTGGCAACAAACCTGACCACATGATTAAGCCTGTTGAAGTCACTGA
GTCAGCATAA
SEQ ID NO: 2 (human O-linked N-acetylglucosamine (GlcNAc) transferase)
MASSVGNVADSTEPTKRMLSFQGLAELAHREYQAGDFEAAERHCMQLWRQEPDN
TGVLLLLSSIHFQCRRLDRSAHFSTLAIKQNPLLAEAYSNLGNVYKERGQLQEAIEH
YRHALRLKPDFIDGYINLAAALVAAGDMEGAVQAYVSALQYNPDLYCVRSDLGNL
LKALGRLEEAKACYLKAIETQPNFAVAWSNLGCVFNAQGEIWLAIHHFEKAVTLDP
NFLDAYINLGNVLKEARIFDRAVAAYLRALSLSPNHAVVHGNLACVYYEQGLIDLAI
DTYRRAIELQPHFPDAYCNLANALKEKGSVAEAEDCYNTALRLCPTHADSLNNLAN
IKREQGNIEEAVRLYRKALEVFPEFAAAHSNLASVLQQQGKLQEALMHYKEAIRISP
TFADAYSNMGNTLKEMQDVQGALQCYTRAIQINPAFADAHSNLASIHKDSGNIPEAI
ASYRTALKLKPDFPDAYCNLAHCLQIVCDWTDYDERMKKLVSIVADQLEKNRLPSV
HPHHSMLYPLSHGFRKAIAERHGNLCLDKINVLHKPPYEHPKDLKLSDGRLRVGYV
SSDFGNHPTSHLMQSIPGMHNPDKFEVFCYALSPDDGTNFRVKVMAEANHFIDLSQI
PCNGKAADRIHQDGIHILVNMNGYTKGARNELFALRPAPIQAMWLGYPGTSGALF
MDYIITDQETSPAEVAEQYSEKLAYMPHTFFIGDHANMFPHLKKKAVIDFKSNGHIY
DNRIVLNGIDLKAFLDSLPDVKIVKMKCPDGGDNADSSNTALNMPVIPMNTIAEAVI
EMINRGQIQITINGFSISNGLATTQINNKAATGEEVPRTIIVTTRSQYGLPEDAIVYCNF
NQLYKIDPSTLQMWANILKRVPNSVLWLLRFPAVGEPNIQQYAQNMGLPQNRIIFSPV
APKEEHVRRGQLADVCLDTPLCNGHTTGMDVLWAGTPMVTMPGETLASRVAASQ
LTCLGCLELIAKNRQEYEDIAVKLGTDLEYLKKVRGKVWKQRISSPLFNTKQYTME
LERLYLQMWEHYAAGNKPDHMIKPVEVTESA
SEQ ID NO: 3 (human OGA-O-GlcNAcase, Gene ID 10724)
ATGGTGCAGAAGGAGAGTCAAGCGACGTTGGAGGAGCGGGAGAGCGAGCTCA
GCTCCAACCCTGCCGCCTCTGCGGGGGCATCGCTGGAGCCGCCGGCAGCTCCGG
CACCCGGAGAAGACAACCCCGCCGGGGCTGGGGGAGCGGCGGTGGCCGGGGCT
GCAGGAGGGGCTCGGCGGTTCCTCTGCGGTGTGGTGGAAGGATTTTATGGAAGA
CCTTGGGTTATGGAACAGAGAAAAGAACTCTTTAGAAGGCTCCAGAAATGGGAA
TTAAATACATACTTGTATGCCCCAAAAGATGACTACAAACATAGGATGTTTTGGCG
AGAGATGTATTCAGTGGAGGAAGCTGAGCAACTTATGACTCTCATCTCTGCTGCA
CGAGAATATGAGATAGAGTTCATCTATGCGATCTCACCTGGATTGGATATCACTTT
TTCTAACCCCAAGGAAGTATCCACATTGAAACGTAAATTGGACCAGGTTTCTCAG
TTTGGGTGCAGATCATTTGCTTTGCTTTTTGATGATATAGACCATAATATGTGTGCA
GCAGACAAAGAGGTATTCAGTTCTTTTGCTCATGCCCAAGTCTCCATCACAAATG
AAATCTATCAGTACCTAGGAGAGCCAGAAACTTTCCTCTTCTGTCCCACAGAATA
CTGTGGCACTTTCTGTTATCCAAATGTGTCTCAGTCTCCATATTTAAGGACTGTGG
GTGAAAAGCTTCTACCTGGAATTGAAGTGCTTTGGACAGGTCCCAAAGTTGTTT
CTAAAGAAATTCCAGTAGAGTCCATCGAAGAGGTTTCTAAGATTATTAAGAGAGC
TCCAGTAATCTGGGATAACATTCATGCTAATGATTATGATCAGAAGAGACTGTTTC
TGGGCCCGTACAAAGGAAGATCCACAGAACTCATCCCACGGTTAAAAGGAGTCC
TCACTAATCCAAATTGTGAATTTGAAGCCAACTACGTTGCTATCCACACCCTTGC
CACCTGGTACAAATCAAACATGAATGGAGTGAGAAAAGATGTAGTGATGACTGA
CAGTGAAGATAGTACTGTGTCCATCCAGATAAAATTAGAAAATGAAGGCAGTGAT
GAAGATATTGAAACTGATGTACTCTATAGTCCACAGATGGCTCTAAAGCTAGCATT
AACAGAATGGTTGCAAGAGTTTGGTGTGCCTCATCAATACAGCAGTAGGCAAGT
TGCACACAGTGGAGCTAAAGCAAGTGTAGTTGATGGGACTCCTTTAGTTGCAGC
ACCCTCTTTAAATGCCACAACCGTAGTAACAACAGTTTATCAGGAGCCCATTATG
AGCCAGGGAGCAGCCTTGAGTGGTGAGCCTACTACTCTGACCAAGGAAGAAGA
AAAGAAACAGCCTGATGAAGAACCCATGGACATGGTGGTGGAAAAACAAGAAG
AAACGGACCACAAGAATGACAATCAAATACTGAGTGAAATTGTTGAAGCGAAAA
TGGCAGAGGAATTGAAACCAATGGACACTGATAAAGAGAGCATAGCTGAATCAA
AATCCCCAGAGATGTCCATGCAAGAAGATTGTATTAGTGACATTGCCCCCATGCA
AACTGATGAACAGACAAACAAGGAGCAGTTTGTGCCAGGTCCAAATGAAAAGC
CTTTGTACACTGCGGAACCAGTGACCCTGGAGGATTTGCAGTTACTTGCTGATCT
ATTCTACCTTCCTTACGAGCATGGACCCAAAGGAGCACAGATGTTACGGGAATTT
CAATGGCTTCGAGCAAATAGTAGTGTTGTCAGTGTCAATTGCAAAGGAAAAGAC
TCTGAAAAAATTGAAGAATGGCGGTCACGAGCAGCCAAGTTTGAAGAGATGTGT
GGACTAGTGATGGGAATGTTCACTCGGCTCTCCAATTGTGCCAACAGGACAATTC
TTTATGACATGTACTCCTATGTTTGGGATATCAAGAGTATAATGTCTATGGTGAAGT
CTTTTGTACAGTGGTTAGGGTGTCGTAGTCATTCTTCAGCACAATTCTTAATTGGA
GACCAAGAACCCTGGGCCTTTAGAGGTGGTCTAGCAGGAGAGTTCCAGCGTTTG
CTGCCAATTGATGGGGCAAATGATCTCTTTTTTCAGCCACCTCCACTGACTCCTA
CCTCCAAAGTTTATACTATCAGACCTTATTTTCCTAAGGATGAGGCATCCGTGTAC
AAGATTTGCAGAGAAATGTATGACGATGGAGTGGGTTTACCCTTTCAAAGTCAGC
CTGATCTTATTGGAGACAAGTTAGTAGGAGGGCTGCTTTCCCTCAGCCTGGATTA
CTGCTTTGTCCTAGAAGATGAAGATGGCATATGTGGTTATGCCTTGGGCACTGTA
GATGTGACCCCCTTTATTAAAAAATGTAAAATTTCCTGGATCCCCTTCATGCAGGA
GAAGTATACCAAGCCAAATGGTGACAAGGAACTCTCTGAGGCTGAGAAAATAAT
GTTGAGTTTCCATGAAGAACAGGAAGTACTGCCAGAAACTTTCCTTGCTAATTTC
CCTTCTCTGATAAAGATGGACATTCACAAAAAAGTAACTGACCCAAGTGTGGCC
AAAAGCATGATGGCTTGCCTCCTGTCTTCACTGAAGGCTAATGGCTCCCGGGGA
GCTTTCTGTGAAGTGAGACCAGATGATAAAAGAATTCTGGAATTTTACAGCAAGT
TAGGATGTTTTGAAATTGCAAAAATGGAAGGATTTCCAAAGGATGTGGTTATACT
TGGTCGGAGCCTGTGA
SEQ ID NO: 4 (human OGA-O-GlcNAcase, Gene ID 10724)
MVQKESQATLEERESELSSNPAASAGASLEPPAAPAPGEDNPAGAGGAAVAGAAGG
ARRFLCGVVEGFYGRPWVMEQRKELFRRLQKWELNTYLYAPKDDYKHRMFWRE
MYSVEEAEQLMTLISAAREYEIEFIYAISPGLDITFSNPKEVSTLKRKLDQVSQFGCRS
FALLFDDIDHNMCAADKEVFSSFAHAQVSITNEIYQYLGEPETFLFCPTEYCGTFCYP
NVSQSPYLRTVGEKLLPGIEVLWTGPKVVSKEIPVESIEEVSKIIKRAPVIWDNIHAN
DYDQKRLFLGPYKGRSTELIPRLKGVLTNPNCEFEANYVAIHTLATWYKSNMNGVR
KDVVMTDSEDSTVSIQIKLENEGSDEDIETDVLYSPQMALKLALTEWLQEFGVPHQ
YSSRQVAHSGAKASVVDGTPLVAAPSLNATTVVTTVYQEPIMSQGAALSGEPTTLTK
EEEKKQPDEEPMDMVVEKQEETDHKNDNQILSEIVEAKMAEELKPMDTDKESIAES
KSPEMSMQEDCISDIAPMQTDEQTNKEQFVPGPNEKPLYTAEPVTLEDLQLLADLFY
LPYEHGPKGAQMLREFQWLRANSSVVSVNCKGKDSEKIEEWRSRAAKFEEMCGLV
MGMFTRLSNCANRTILYDMYSYVWDIKSIMSMVKSFVQWLGCRSHSSAQFLIGDQ
EPWAFRGGLAGEFQRLLPIDGANDLFFQPPPLTPTSKVYTIRPYFPKDEASVYKICRE
MYDDGVGLPFQSQPDLIGDKLVGGLLSLSLDYCFVLEDEDGICGYALGTVDVTPFIK
KCKISWIPFMQEKYTKPNGDKELSEAEKIMLSFHEEQEVLPETFLANFPSLIKMDIHK
KVTDPSVAKSMMACLLSSLKANGSRGAFCEVRPDDKRILEFYSKLGCFEIAKMEGF
PKDVVILGRSL

1. A method for vascular regeneration in a subject with a peripheral vascular disease, the method comprising:
administering an effective amount of an O-glycnacylation modifier agent to an injured peripheral vascular tissue in the subject.

2. The method of claim 1, further comprising administering an effective amount of an inflammation agent inducer, an angiogenic factor, or any combination thereof to the wound of the subject.

3. The method of claim 1, wherein the method promotes vascular regeneration in the injured peripheral vascular tissue by at least 30% compared to the peripheral vascular tissue without administration of an effective amount of an O-glycnacylation modifier agent, as determined by laser doppler perfusion.

4. The method of claim 1, wherein the method increases O-glycnacylation level in the injured peripheral vascular tissue compared to O-glycnacylation level without administration of an effective amount of an O-glycnacylation modifier agent.

5. The method of claim 1, wherein the method increases the concentration O-GlycNAC transferase (OGT) in the injured peripheral vascular tissue compared to the concentration O-GlycNAC transferase (OGT) without administration of an effective amount of an O-glycnacylation modifier agent.

6. The method of claim 1, wherein the method inhibits O-GlycNACase (OGA) in the injured peripheral vascular tissue compared to the O-GlycNACase (OGA) level without administration of an effective amount of an O-glycnacylation modifier agent.

7. The method of claim 1, wherein the peripheral vascular disease comprises peripheral arterial disease, limb ischemia, popliteal entrapment syndrome, Raynaud's disease, Buerger's disease, or any combination thereof.

8. The method of claim 1, wherein the peripheral vascular disease is peripheral arterial occlusive disease.

9. The method of claim 1, wherein the peripheral vascular disease is associated with limb ischemia.

10. The method of claim 1, wherein the injured peripheral vascular tissue comprises cutaneous tissue, epithelial tissue, connective tissue, muscle tissue, bone, nervous tissue, or any combination thereof.

11. A method of treating a wound in a subject in need thereof, the method comprising:
administering an effective amount of an O-glycnacylation modifier agent to the wound in the subject.

12. The method of claim 11, further comprising administering an effective amount of an inflammation agent inducer, an angiogenic factor, or any combination thereof to the wound in the subject.

13. The method of claim 11, wherein the method promotes wound healing by an amount of from 5% to 50% compared to the wound healing without administration of an effective amount of an O-glycnacylation modifier agent, as determined by digital photography and planimetry.

14. The method of claim 11, wherein the method increases O-glycnacylation level in the wound compared to O-glycnacylation level without administration of an effective amount of an O-glycnacylation modifier agent.

15. The method of claim 11, wherein the method increases the concentration of O-GlycNAC transferase (OGT) in the wound compared to the concentration O-GlycNAC transferase (OGT) without administration of an effective amount of an O-glycnacylation modifier agent.

16. The method of claim 11, wherein the method inhibits O-GlycNACase (OGA) in the wound compared to the O-GlycNACase (OGA) level without administration of an effective amount of an O-glycnacylation modifier agent.

17. The method of claim 11, wherein the wound exhibits delayed healing.

18. The method of claim 11, wherein the wound comprises abdominal wounds or other large or incisional wounds, dehisced wounds, acute wounds, chronic wounds, subacute and dehisced wounds, traumatic wounds, vascular wounds, flaps and skin grafts, surgical wounds, lacerations, abrasions, contusions, hematomas, burns, diabetic ulcers, pressure ulcers, stoma, cosmetic wounds, trauma ulcers, neuropathic ulcers, venous and arterial ulcers, chronic or non-healing wounds, or any combination thereof.

19. The method of claim 11, wherein the wound comprises a vascular wound.

20. The method of claim 1, wherein the O-glycnacylation modifier agent comprises 2-(ethylamino)-5-(hydroxymethyl)-5,6,7,7a-tetrahydro-3aH-pyrano[3,2-d][1,3]thiazole-6,7-diol (TMG); (3aR,5R,6S,7R,7aR)-3a,6,7,7a-Tetrahydro-5-(hydroxymethyl)-2-propyl-5H-pyrano[3,2-d]thiazole-6,7-diol (NButGT); NAG-thiazoline (i.e. 2 ‘-methyl-a D-glucopyrano-[2,1-i/]-A2’-thiazoline); O-(2-acetamido-2-deoxy-D-glucopyranosylidene)amino-Z—N-phenylcarbamate) (PUGNAc); a polynucleotide sequence encoding O-GlycNAC transferase (OGT), a fragment, or variant thereof; uridine diphosphate N-acetylglucosamine (UDP-GlcNAc); glucose; glutamine; glucosamine; or any combination thereof.

21. The method of claim 1, wherein the O-glycnacylation modifier agent comprises 2-(ethylamino)-5-(hydroxymethyl)-5,6,7,7a-tetrahydro-3aH-pyrano[3,2-d][1,3]thiazole-6,7-diol (TMG).

22. The method of claim 1, wherein the angiogenic factor comprises VEGF, fibroblast growth factor, hypoxia-inducible growth factor, platelet-derived growth factor, bone matrix protein 4, angiopoeitins, nitric oxide or other agents that increase intracellular cGMP, prostacyclin or other agents that increase intracellular cAMP, or any combination thereof.

23. The method of claim 1, wherein the inflammation agent inducer comprises TLR3 agonist polyinosinic:polycytidilic acid (PolyIC), inflammatory cytokines such as interleukins IL-1a, IL-6 or IL-8, lipopolysaccharide (LPS) or lipoteichoic acid (LTA), tumor necrosis factor alpha, or any combination thereof.

24. The method of claim 20, wherein the polynucleotide sequence encodes O-GlycNAC transferase, a fragment, or a variant thereof comprising an amino acid sequence with at least 80% sequence identity to SEQ ID NO: 2.

25. The method of claim 20, wherein the polynucleotide sequence encodes O-GlycNAC transferase, a fragment, or a variant thereof comprising an amino acid sequence with at least 95% sequence identity to SEQ ID NO: 2.

26. The method of claim 20, wherein the polynucleotide sequence encoding O-GlycNAC transferase (OGT) comprises modified nucleosides that increase translational efficiency and/or reduce immunogenicity.

27. The method of claim 20, wherein the polynucleotide sequence is a DNA sequence.

28. The method of claim 27, wherein the DNA sequence comprises a coding region encoding O-GlycNAC transferase, a fragment, or a variant thereof, wherein the DNA sequence comprises a sequence with at least 80% sequence identity to SEQ ID NO: 1.

29. The method of claim 27, wherein the DNA sequence comprises a coding region encoding O-GlycNAC transferase, a fragment, or a variant thereof, wherein the DNA sequence comprises a sequence with at least 95% sequence identity to SEQ ID NO: 1.

30. The method of claim 28, wherein the O-GlycNAC transferase, a fragment, or a variant thereof comprises an amino acid sequence with at least 80% sequence identity to SEQ ID NO: 2.

31. The method of claim 28, wherein the O-GlycNAC transferase, a fragment, or a variant thereof comprises an amino acid sequence with at least 95% sequence identity to SEQ ID NO: 2.

32. The method of claim 28, wherein the DNA encoding O-GlycNAC transferase (OGT) is circular.

33. The method of claim 1, wherein administration comprises topical, intravenous, subcutaneous, transcutaneous, transdermal, intramuscular, intradermal, intra-arteriole, intralesional, or any combination thereof.


SEQUENCE LISTING (4 sequences)

SEQ ID NO: 1 - DNA, 3141 bp, Homo sapiens
  source 1..3141: mol_type = genomic DNA
atggcgtcttccgtgggcaacgtggccgacagcacagaaccaacgaaacgtatgctttccttccaagggttagctgagttggcacatcgagaatatcaggcaggagattttgaggcagctgagagacactgcatgcagctctggagacaagagccagacaatactggtgtgcttttattactttcatctatacacttccagtgtcgaaggctggacagatctgctcactttagcactctggcaattaaacagaacccccttctggcagaagcttattcgaatttggggaatgtgtacaaggaaagagggcagttgcaggaggcaattgagcattatcgacatgcattgcgtctcaaacctgatttcatcgatggttatattaacctggcagccgccttggtagcagcgggtgacatggaaggggcagtacaagcttacgtctctgctcttcagtacaatcctgatttgtactgtgttcgcagtgacctggggaacctgctcaaagccctgggtcgcttggaagaagccaaggcatgttatttgaaagcaattgagacgcaaccgaactttgcagtagcttggagtaatcttggctgtgttttcaatgcacaaggggaaatttggcttgcaattcatcactttgaaaaggctgtcacccttgacccaaactttctggatgcttatatcaatttaggaaatgtcttgaaagaggcacgcatttttgacagagctgtggcagcttatcttcgtgccctaagtttgagtccaaatcacgcagtggtgcacggcaacctggcttgtgtatactatgagcaaggcctgatagatctggcaatagacacctacaggcgggctatcgaactacaaccacatttccctgatgcttactgcaacctagccaatgctctcaaagagaagggcagtgttgctgaagcagaagattgttataatacagctctccgtctgtgtcccacccatgcagactctctgaataacctagccaatatcaaacgagaacagggaaacattgaagaggcagttcgcttgtatcgtaaagcattagaagtcttcccagagtttgctgctgcccattcaaatttagcaagtgtactgcagcagcagggaaaactgcaggaagctctgatgcattataaggaggctattcgaatcagtcctacctttgctgatgcctactctaatatgggaaacactctaaaggagatgcaggatgttcagggagccttgcagtgttatacgcgtgccatccaaattaatcctgcatttgcagatgcacatagcaatctggcttccattcataaggattcagggaatattccagaagccatagcttcttaccgcacggctctgaaacttaagcctgattttcctgatgcttattgtaacttggctcattgcctgcagattgtctgtgattggacagactatgatgagcgaatgaagaagttggtcagtattgtggctgaccagttagagaagaataggttgccttctgtgcatcctcatcatagtatgctatatcctctttctcatggcttcaggaaggctattgctgagaggcacggcaacctgtgcttagataagattaatgttcttcataaaccaccatatgaacatccaaaagacttgaagctcagtgatggtcggctgcgtgtaggatatgtgagttccgactttgggaatcatcctacttctcaccttatgcagtctattccaggcatgcacaatcctgataaatttgaggtgttctgttatgccctgagcccagacgatggcacaaacttccgagtgaaggtgatggcagaagccaatcatttcattgatctttctcagattccatgcaatggaaaagcagctgatcgcatccatcaggatggaattcatatccttgtaaatatgaatggctatactaagggcgctcgaaatgagctttttgctctcaggccagctcctattcaggcaatgtggctgggataccctgggacgagtggtgcgcttttcatggattatattatcactgatcaggaaacttcgccagctgaagttgctgagcagtattccgagaaattggcttatatgccccacactttttttattggtgatcatgctaatatgttccctcacctgaagaaaaaagcagtcatcgattttaagtccaatgggcacatttatgacaatcggatagttctgaatggcatcgacctcaaagcatttcttgatagtctaccagatgtgaaaattgtcaagatgaagtgtcctgatggaggagacaatgcagatagcagtaacacagctcttaatatgcctgttattcctatgaatactattgcagaagcagttattgaaatgattaaccgaggacagattcaaataacaattaatggattcagtattagcaatggactggcaactactcagatcaacaataaggctgcaactggagaggaggttccccgtaccattattgtaaccacccgttctcagtacgggttaccagaagatgccatcgtatactgtaactttaatcagttgtataaaattgacccttctactttgcagatgtgggcaaacattctgaagcgtgttcccaatagtgtactctggctgttgcgttttccagcagtaggagaacctaatattcaacagtatgcacaaaacatgggcctgccccagaaccgtatcattttttcacctgttgctcctaaagaggaacacgtcaggagaggccagctggctgatgtctgcttggacactccactctgtaatgggcacaccacagggatggatgtcctctgggcagggacccccatggtgactatgccaggagagactcttgcttctcgagttgcagcatcccagctcacttgcttaggttgtcttgagcttattgctaaaaacagacaagaatatgaagacatagctgtgaagctgggaactgatctagaatacctgaagaaagttcgtggcaaagtctggaagcaaagaatatctagccctctgttcaacaccaaacaatacacaatggaactagagcggctctatctacagatgtgggagcattatgcagctggcaacaaacctgaccacatgattaagcctgttgaagtcactgagtcagcataa

SEQ ID NO: 2 - protein, 1046 aa, Homo sapiens
MASSVGNVADSTEPTKRMLSFQGLAELAHREYQAGDFEAAERHCMQLWRQEPDNTGVLLLLSSIHFQCRRLDRSAHFSTLAIKQNPLLAEAYSNLGNVYKERGQLQEAIEHYRHALRLKPDFIDGYINLAAALVAAGDMEGAVQAYVSALQYNPDLYCVRSDLGNLLKALGRLEEAKACYLKAIETQPNFAVAWSNLGCVFNAQGEIWLAIHHFEKAVTLDPNFLDAYINLGNVLKEARIFDRAVAAYLRALSLSPNHAVVHGNLACVYYEQGLIDLAIDTYRRAIELQPHFPDAYCNLANALKEKGSVAEAEDCYNTALRLCPTHADSLNNLANIKREQGNIEEAVRLYRKALEVFPEFAAAHSNLASVLQQQGKLQEALMHYKEAIRISPTFADAYSNMGNTLKEMQDVQGALQCYTRAIQINPAFADAHSNLASIHKDSGNIPEAIASYRTALKLKPDFPDAYCNLAHCLQIVCDWTDYDERMKKLVSIVADQLEKNRLPSVHPHHSMLYPLSHGFRKAIAERHGNLCLDKINVLHKPPYEHPKDLKLSDGRLRVGYVSSDFGNHPTSHLMQSIPGMHNPDKFEVFCYALSPDDGTNFRVKVMAEANHFIDLSQIPCNGKAADRIHQDGIHILVNMNGYTKGARNELFALRPAPIQAMWLGYPGTSGALFMDYIITDQETSPAEVAEQYSEKLAYMPHTFFIGDHANMFPHLKKKAVIDFKSNGHIYDNRIVLNGIDLKAFLDSLPDVKIVKMKCPDGGDNADSSNTALNMPVIPMNTIAEAVIEMINRGQIQITINGFSISNGLATTQINNKAATGEEVPRTIIVTTRSQYGLPEDAIVYCNFNQLYKIDPSTLQMWANILKRVPNSVLWLLRFPAVGEPNIQQYAQNMGLPQNRIIFSPVAPKEEHVRRGQLADVCLDTPLCNGHTTGMDVLWAGTPMVTMPGETLASRVAASQLTCLGCLELIAKNRQEYEDIAVKLGTDLEYLKKVRGKVWKQRISSPLFNTKQYTMELERLYLQMWEHYAAGNKPDHMIKPVEVTESA

SEQ ID NO: 3 - DNA, 2751 bp, Homo sapiens
  source 1..2751: mol_type = genomic DNA
atggtgcagaaggagagtcaagcgacgttggaggagcgggagagcgagctcagctccaaccctgccgcctctgcgggggcatcgctggagccgccggcagctccggcacccggagaagacaaccccgccggggctgggggagcggcggtggccggggctgcaggaggggctcggcggttcctctgcggtgtggtggaaggattttatggaagaccttgggttatggaacagagaaaagaactctttagaaggctccagaaatgggaattaaatacatacttgtatgccccaaaagatgactacaaacataggatgttttggcgagagatgtattcagtggaggaagctgagcaacttatgactctcatctctgctgcacgagaatatgagatagagttcatctatgcgatctcacctggattggatatcactttttctaaccccaaggaagtatccacattgaaacgtaaattggaccaggtttctcagtttgggtgcagatcatttgctttgctttttgatgatatagaccataatatgtgtgcagcagacaaagaggtattcagttcttttgctcatgcccaagtctccatcacaaatgaaatctatcagtacctaggagagccagaaactttcctcttctgtcccacagaatactgtggcactttctgttatccaaatgtgtctcagtctccatatttaaggactgtgggtgaaaagcttctacctggaattgaagtgctttggacaggtcccaaagttgtttctaaagaaattccagtagagtccatcgaagaggtttctaagattattaagagagctccagtaatctgggataacattcatgctaatgattatgatcagaagagactgtttctgggcccgtacaaaggaagatccacagaactcatcccacggttaaaaggagtcctcactaatccaaattgtgaatttgaagccaactacgttgctatccacacccttgccacctggtacaaatcaaacatgaatggagtgagaaaagatgtagtgatgactgacagtgaagatagtactgtgtccatccagataaaattagaaaatgaaggcagtgatgaagatattgaaactgatgtactctatagtccacagatggctctaaagctagcattaacagaatggttgcaagagtttggtgtgcctcatcaatacagcagtaggcaagttgcacacagtggagctaaagcaagtgtagttgatgggactcctttagttgcagcaccctctttaaatgccacaaccgtagtaacaacagtttatcaggagcccattatgagccagggagcagccttgagtggtgagcctactactctgaccaaggaagaagaaaagaaacagcctgatgaagaacccatggacatggtggtggaaaaacaagaagaaacggaccacaagaatgacaatcaaatactgagtgaaattgttgaagcgaaaatggcagaggaattgaaaccaatggacactgataaagagagcatagctgaatcaaaatccccagagatgtccatgcaagaagattgtattagtgacattgcccccatgcaaactgatgaacagacaaacaaggagcagtttgtgccaggtccaaatgaaaagcctttgtacactgcggaaccagtgaccctggaggatttgcagttacttgctgatctattctaccttccttacgagcatggacccaaaggagcacagatgttacgggaatttcaatggcttcgagcaaatagtagtgttgtcagtgtcaattgcaaaggaaaagactctgaaaaaattgaagaatggcggtcacgagcagccaagtttgaagagatgtgtggactagtgatgggaatgttcactcggctctccaattgtgccaacaggacaattctttatgacatgtactcctatgtttgggatatcaagagtataatgtctatggtgaagtcttttgtacagtggttagggtgtcgtagtcattcttcagcacaattcttaattggagaccaagaaccctgggcctttagaggtggtctagcaggagagttccagcgtttgctgccaattgatggggcaaatgatctcttttttcagccacctccactgactcctacctccaaagtttatactatcagaccttattttcctaaggatgaggcatccgtgtacaagatttgcagagaaatgtatgacgatggagtgggtttaccctttcaaagtcagcctgatcttattggagacaagttagtaggagggctgctttccctcagcctggattactgctttgtcctagaagatgaagatggcatatgtggttatgccttgggcactgtagatgtgaccccctttattaaaaaatgtaaaatttcctggatccccttcatgcaggagaagtataccaagccaaatggtgacaaggaactctctgaggctgagaaaataatgttgagtttccatgaagaacaggaagtactgccagaaactttccttgctaatttcccttctctgataaagatggacattcacaaaaaagtaactgacccaagtgtggccaaaagcatgatggcttgcctcctgtcttcactgaaggctaatggctcccggggagctttctgtgaagtgagaccagatgataaaagaattctggaattttacagcaagttaggatgttttgaaattgcaaaaatggaaggatttccaaaggatgtggttatacttggtcggagcctgtga

SEQ ID NO: 4 - protein, 916 aa, Homo sapiens
MVQKESQATLEERESELSSNPAASAGASLEPPAAPAPGEDNPAGAGGAAVAGAAGGARRFLCGVVEGFYGRPWVMEQRKELFRRLQKWELNTYLYAPKDDYKHRMFWREMYSVEEAEQLMTLISAAREYEIEFIYAISPGLDITFSNPKEVSTLKRKLDQVSQFGCRSFALLFDDIDHNMCAADKEVFSSFAHAQVSITNEIYQYLGEPETFLFCPTEYCGTFCYPNVSQSPYLRTVGEKLLPGIEVLWTGPKVVSKEIPVESIEEVSKIIKRAPVIWDNIHANDYDQKRLFLGPYKGRSTELIPRLKGVLTNPNCEFEANYVAIHTLATWYKSNMNGVRKDVVMTDSEDSTVSIQIKLENEGSDEDIETDVLYSPQMALKLALTEWLQEFGVPHQYSSRQVAHSGAKASVVDGTPLVAAPSLNATTVVTTVYQEPIMSQGAALSGEPTTLTKEEEKKQPDEEPMDMVVEKQEETDHKNDNQILSEIVEAKMAEELKPMDTDKESIAESKSPEMSMQEDCISDIAPMQTDEQTNKEQFVPGPNEKPLYTAEPVTLEDLQLLADLFYLPYEHGPKGAQMLREFQWLRANSSVVSVNCKGKDSEKIEEWRSRAAKFEEMCGLVMGMFTRLSNCANRTILYDMYSYVWDIKSIMSMVKSFVQWLGCRSHSSAQFLIGDQEPWAFRGGLAGEFQRLLPIDGANDLFFQPPPLTPTSKVYTIRPYFPKDEASVYKICREMYDDGVGLPFQSQPDLIGDKLVGGLLSLSLDYCFVLEDEDGICGYALGTVDVTPFIKKCKISWIPFMQEKYTKPNGDKELSEAEKIMLSFHEEQEVLPETFLANFPSLIKMDIHKKVTDPSVAKSMMACLLSSLKANGSRGAFCEVRPDDKRILEFYSKLGCFEIAKMEGFPKDVVILGRSL